(19) United States
(12) Patent Application Publication
(10) Pub. No.: US 2010/0112561 A1
(43) Pub. Date: May 6, 2010

(54) FLUORESCENT NUCLEOSIDE ANALOGUES

(76) Inventors: Stefan Lutz, Tucker, GA (US); Dennis Liotta, Atlanta, GA (US); Yongfeng Li, Marietta, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/310,455

(22) PCT Filed: Aug. 27, 2007
(86) PCT No.: PCT/US07/18883

§ 371 Date: Oct. 7, 2009

Related U.S. Application Data
(60) Provisional application No. 60/840,321, filed on Aug. 25, 2006.

(51) Int. Cl. C12Q 1/68; C07H 19/06; C07H 19/00; C07H 19/22

(52) U.S. Cl. 435/6; 536/28.4; 536/27.2; 536/27.13

(57) ABSTRACT

Briefly described, embodiments of the present disclosure include novel fluorescent nucleoside analogs (fNAs) including a fluorescent nucleobase, selected from a purine and a pyrimidine base or analog thereof, and a modified sugar moiety that differs in structure from a sugar moiety of a naturally occurring nucleoside. In embodiments, the fNAs of the present disclosure are analogues of NA prodrugs used to treat viral disorders. Embodiments of the present disclosure also include methods of making the novel fNAs of the present disclosure.

### CROSS-REFERENCE TO RELATED APPLICATION

This application claims priority to U.S. provisional application entitled, “FLUORESCENT NUCLEOSIDE ANALOGUES,” having Ser. No. 60/840,321, filed on Aug. 25, 2006, which is entirely incorporated herein by reference.

### STATEMENT REGARDING FEDERALLY SPONSORED RESEARCH OR DEVELOPMENT

This invention was made in part with government support under grant number GM 69958 awarded by the NIH. The government has certain rights in the invention.

### BACKGROUND

Viral diseases, as well as the recent threat of weaponized viruses, represent a continuous global health problem. Worldwide, more that 50 million people have become infected with HIV since its emergence twenty years ago, and the annual death toll has risen to 2.5 million. In 2005, the WHO estimated 350 million cases of chronic hepatitis B and 170 million cases of hepatitis C infection, both of which include cirrhosis and hepatocellular carcinoma as long-term consequences. More recently, the importance of antiviral drugs as last line of defense has been highlighted by vaccine shortages and efficiency concerns, given the limitations to predict future viral variants. These concerns not only include the annual influenza wave but the threat of viruses when used as biological weapons (e.g., engineered smallpox and Ebola virus). Over the years, several generations of antiviral drugs have been introduced, targeting viral proteins involved in host cell entry and genomic integration, as well as DNA replication and proteolytic processing of viral precursor proteins.

The most dominant group of antiviral drugs are nucleoside analogues (NAs). From a functional perspective, NAs are actually prodrugs whose bioavailability depends on intracellular phosphorylation to the triphosphate. Administered in their uncharged nucleosidic form, these compounds utilize the host's nucleoside salvage pathway, as illustrated in FIG. 1, for membrane passage and subsequent sequential phosphorylation by deoxynucleoside kinases (dNK) (e.g., thymidine kinase), deoxynucleotide monophosphate kinases (dNMPK) (e.g., thymidylate kinase), and deoxynucleotide diphosphate kinase (dNDP kinase). Resembling the natural building blocks of DNA and RNA, the triphosphate anabolites then turn into competitive substrates for a virus' low-fidelity polymerase or reverse transcriptase. The incorporation of NA-triphosphates results in the immediate termination of the replication process, preventing further viral proliferation. The mammalian cell's replication machinery on the other hand has a higher fidelity, protecting the host from the lethal effects of these suicide substrates.

While theoretically sound, this process is flawed in practice by the NA's dependence on the phosphorylative activation by the cellular nucleoside and nucleotide kinases. While nucleoside transport proteins show relatively broad specificity for nucleosides and NAs, the high substrate specificity of the human nucleoside and nucleotide kinases reduces the effective turnover of many prodrugs. In particular, the two initial phosphorylation reactions by human dNKs and dNMPKs limit triphosphate formation, which results in the accumulation of NAs and NA monophosphates inside the cell. Consequently, many prodrug candidates that show promising activity in primer extension experiments in vitro fail to express measurable effects in vivo.

The stage at which NAs build up varies between individual analogues. For instance, 2′,3′-didehydro-2′,3′-dideoxy thymidine (d4T) is a poor substrate for dNKs and accumulates as the nucleoside, while 3′-azido-thymidine (AZT) is turned over to the monophosphate (AZTMP) but can not effectively be phosphorylated by the cell's dNMPKs. Making matters worse, the accumulation of precursor not only lessens the effectiveness of NAs but can actually trigger an adverse cellular response. Drug-induced expression of cellular multidrug resistant protein that actively exports the nucleosidic prodrug out of the cell has been observed. As for AZTMP, the buildup of this intermediate has been shown to interfere with the host metabolism, suppressing kinase activity and possibly causing cytotoxic effects through AZT metabolism to the 3′-amino derivative.

Dramatic effects on cellular metabolism have been observed upon long-term drug treatment. In response to extended exposure to NAs, phenotypic (temporary) and genotypic (permanent) changes were detected in the host cells. A link between declining gene expression levels for dNKs and DNA methylation has been proposed, slowing NA activation and further raising the concentration levels of the intermediates. In addition, studies of high NA levels in mammalian cell cultures and animals suggest a link between NAs and genotoxic effects in the host. In summary, the inefficient phosphorylation of NAs by cellular kinases is a significant contributor to the serious side effects associated with NA-based antiviral treatment.

### SUMMARY

Briefly described, embodiments of the present disclosure include novel fluorescent nucleoside analogs (fNAs) including a fluorescent nucleobase, selected from a purine and a pyrimidine base or analog thereof, and a modified sugar moiety that differs in structure from a sugar moiety of a naturally occurring nucleoside. In embodiments, the fNAs of the present disclosure are analogues of NA prodrugs used to treat viral disorders. Embodiments of the present disclosure also include methods of making the novel fNAs of the present disclosure.

Exemplary embodiments of novel fNAs of the present disclosure include, but are not limited to, a fluorescent nucleobase selected from the nucleobase of one of compounds (1)-(9) (FIGS. 2 and 3) and a modified sugar moiety selected from one of the modified sugar moieties of one of compounds (10)-(16) (FIG. 4). In other words, exemplary embodiments of novel fNAs of the present disclosure include, but are not limited to, compounds having a fluorescent nucleobase selected from one of fluorescent nucleobases (a)-(e) below, where for fluorescent nucleobase (e), X is selected from one of X1-X3, and having a modified sugar moiety selected from one of modified sugar moieties (f)-(l) below, where for nucleobases (a)-(e), R represents one of the modified sugar moieties (f)-(l), and where for modified sugar moieties (f)-(l), R′ represents one of the fluorescent nucleobases (a)-(e).

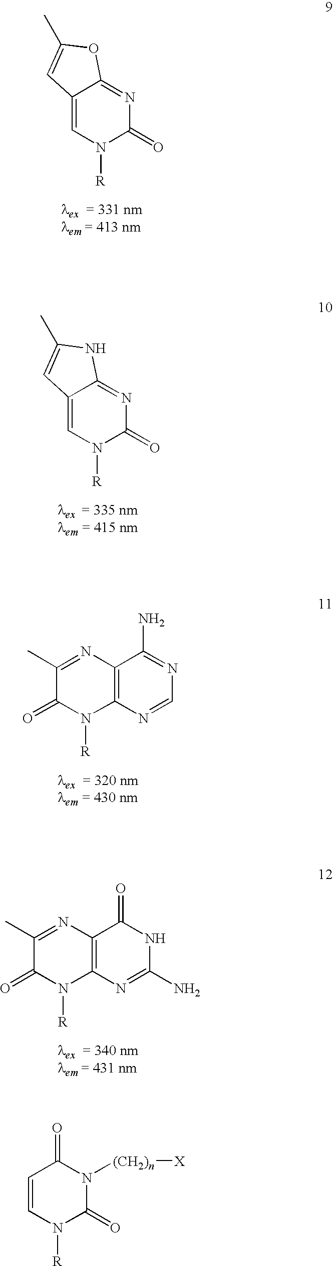

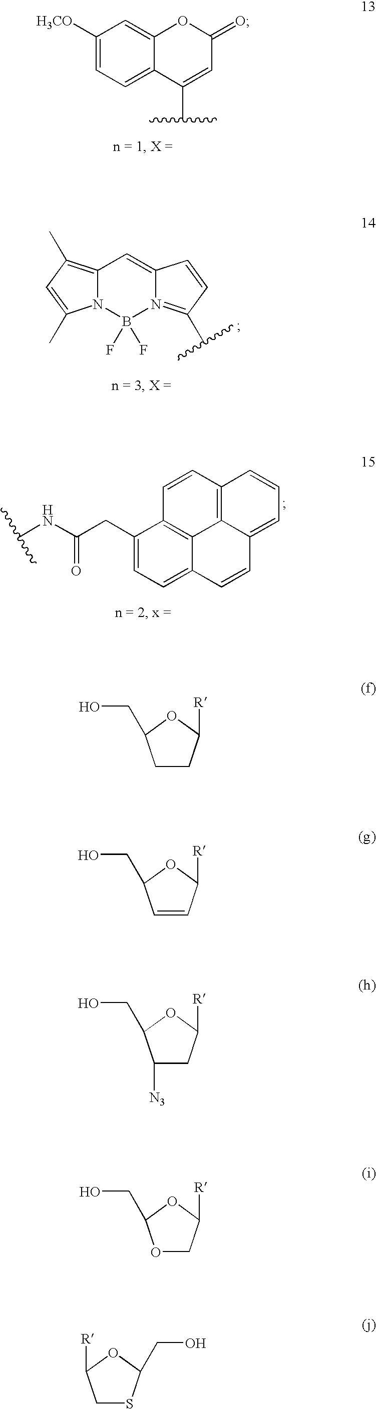

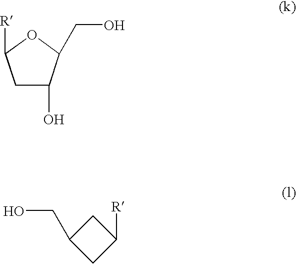

Embodiments of the disclosure also include fNAs including one of the above-modified sugar moieties in combination with a fluorescent nucleobase selected from a purine and a pyrimidine base or an analog thereof. In embodiments, the novel FNAs of the present disclosure include a fluorescent furano-pyrimidine base with a modified sugar moiety. In other embodiments, the fNAs of the present disclosure include a fluorescent pyrrolo-pyrimidine base combined with a modified sugar moiety. In yet other embodiments, the fNAs of the present disclosure include fluorescent pterine bases combined with a modified sugar moiety.

The present disclosure also includes methods of using the fNAs of the present disclosure to detect in vivo phosphorylation of the fNAs including contacting a cell with a solution of fNAs and detecting the change in fluorescence intensity inside the cell. In embodiments, the method includes detecting the accumulation of fNAs inside the cell by detecting an increase in fluorescence intensity in the cell, indicating the phosphorylation of the fNAs. In embodiments the method includes detecting the distribution of the fNAs inside the cell. In some embodiments, fluorescence microscopy is used to detect the change in fluorescence intensity. In some embodiments, a population of cells is contacted with the solution of fNAs, and fluorescence-activated cell sorting (FACS) is used to detect and isolate cells with increased fluorescence.

Embodiments of the present disclosure also include methods of screening for kinases that are able to phosphorylate fNAs including providing one or more cells that over-express a kinase of interest, contacting the cells with a solution of an fNA, and detecting the change in fluorescence intensity inside the cell, whereby an increase in fluorescence intensity indicates phosphorylation of the fNAs by the kinase. In embodiments, the kinase is a deoxynucleoside kinase. In further embodiments, the kinase is a modified deoxynucleoside kinase. In embodiments the method also includes separately contacting one or more cells that over-express the kinase of interest with fluorescent analogs of natural nucleosides, detecting a change in fluorescence intensity inside the cell, and comparing the change in fluorescence intensity to the change in fluorescence intensity detected with the fNA. In embodiments of the methods described above FACS is used to screen large samples of cells for cells with pre-determined levels of fluorescence. Embodiments of the present disclosure also include using the method(s) described above to identify modified kinases with increased activity for nucleoside analogues.

These embodiments, uses of these embodiments, and other uses, features and advantages of the present disclosure, will become more apparent to those of ordinary skill in the relevant art when the following detailed description of the preferred embodiments is read in conjunction with the appended figures.

### BRIEF DESCRIPTION OF THE DRAWINGS

The disclosure can be better understood with reference to the following drawings, described in additional detail in the description and examples below. The components in the drawings are not necessarily to scale, emphasis instead being placed upon clearly illustrating the principles of the present disclosure.

The patent or patent application file contains at least one drawing executed in color. Copies of this patent or patent application publication with color drawing(s) will be provided by the Office upon request and payment of the necessary fee.

FIG. 1 is a schematic illustration of an overview of the nucleoside salvage pathway, exemplified for thymidine.

FIG. 2 illustrates the structures of fluorescent nucleobase compounds 1-6. These exemplary nucleobases are suitable for screening type-I deoxynucleoside kinases.

FIG. 3 illustrates the structures of fluorescent nucleobase compounds 7-9. These exemplary nucleobases are suitable for screening type-II deoxynucleoside kinases.

FIG. 4 illustrates the structures of compounds 10-16. These sugar moieties are exemplary for the type of modifications in the ribose portion of embodiment in this application.

FIG. 5 illustrates the general synthetic strategy for making A) the furano (1) and B) the pyrrolo-pyrimidine (2) nucleobases of a 2′-deoxyribonucleoside as exemplified by 2′-deoxyuridine (17).

FIG. 6 illustrates the synthetic strategy for making the fluorescent nucleoside analog of 2′,3′-dideoxy-thymidine (14) and 2′,3′-dideoxy-cytidine (25), as well as the fluorescent nucleoside analog of 2′,3′-didehydro-2′,3′-dideoxy-thymidine (15) and 2′,3′-didehydro-2′,3′-dideoxy-cytidine (26) from uridine (21).

FIG. 7 illustrates the synthetic strategy for making the fluorescent nucleoside analog of 3′-azido-3′-deoxy-thymidine (16) from 5-iodo-2′-deoxy-uridine (18).

FIG. 8 illustrates digital images of fluorescence microscopy of E. coli KY895 with pDIM-tDmdNK (left) and E. coli TOP10 with pBAD-tDmdNK (right) in the presence of compound 1 (100× magnification; excitation: Hg-lamp at 325 nm). The different morphology is strain-specific and unrelated to the expression of the 2′-deoxyribonucleoside kinase.

FIG. 9 shows histogram illustrating fluorescence-activated cell sorting of E. coli cells expressing genes of 2′-deoxyribonucleoside kinases from different commercial DNA plasmids used for protein overexpression. The graph shows the fluorescence intensity in cell cultures carrying either the pDIM, pBAD, or pET-vectors with the cloned gene for 2′-deoxyribonucleoside kinase from Drosophila melanogaster upon incubation of bacteria with compound 1.

FIG. 10 illustrates a comparison of substrate properties of natural 2′-deoxynucleoside (thymidine; T) and fluorescent analog 1 with various 2′-deoxyribonucleoside kinases. FIG. 10A provides the kinetic data (kcat/KM values), which suggest that the furano-derivative shows the same relative substrate profile as the natural substrate. FIG. 10B illustrates FACS analysis of bacterial cultures overexpressing the various kinases correlating with the kinetic data of FIG. 10A.

FIG. 11 illustrates a comparison of substrate properties of various fluorescent nucleoside analogs (1, 16, 25, 26) with wild type 2′-deoxyribonucleoside kinase from Drosophila melanogaster (wtDmdNK). FIG. 11A provides the kinetic data (kcat/KM values) indicating that the fluorescent nucleoside with the 2′-deoxyribose moiety (1) is a good substrate while nucleoside analogs with modified ribose portions are poor or no substrates for the wild type enzyme, consistent with data for the corresponding natural nucleoside and nucleoside analogs (data not shown). FIG. 11B illustrates FACS analysis of bacterial cultures overexpressing DmdNK and incubated with various fluorescent nucleoside and nucleoside analogs, showing that this analysis correlates well with the kinetic data.

FIG. 12 illustrates graphs comparing artificial mixtures of DmdNK and dCK, two kinases with good (DmdNK) and no activity (dCK) for compound 1. FIG. 12A is a graph showing that incubation of bacteria that overexpress these two kinases, followed by exposure to the fluorescent nucleoside produces a histogram that reflect the various extent of phosphorylation (e.g., a higher ratio of DmdNK in mixture shifts the curve to the right. FIG. 12B) shows that fluorescence activated cell sorting allows for the separation of cells with high fluorescence (in this example >10 units on x-axis). Secondary analysis shows a greater than 100-fold enrichment in cells that carry the DmdNK gene.

### DETAILED DESCRIPTION

Embodiments of the present disclosure will employ, unless otherwise indicated, conventional techniques of synthetic organic chemistry, biochemistry, molecular biology, and the like, which are within the skill of one in the art. Such techniques are explained fully in the literature.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C., and pressure is at or near atmospheric. Standard temperature and pressure are defined as 20° C. and 1 atmosphere.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Before the embodiments of the present disclosure are described in detail, it is to be understood that unless otherwise indicated the present disclosure is not limited to particular materials, reagents, reaction materials, manufacturing processes, or the like, as such may vary. It is also to be understood that the terminology used herein is for purposes of describing particular embodiments only, and is not intended to be limiting. It is also possible in the present disclosure that steps may be executed in different sequence where this is logically possible.

Prior to describing the various embodiments, the following definitions are provided and should be used unless otherwise indicated.

### DEFINITIONS

In describing and claiming the disclosed subject matter, the following terminology will be used in accordance with the definitions set forth below. Unless defined otherwise, all technical and scientific terms used herein have the same meaning as commonly understood by one of ordinary skill in the art to which this disclosure belongs. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted. Although any methods and materials similar or equivalent to those described herein can also be used in the practice or testing of the present disclosure, the preferred methods and materials are described herein.

A “nucleoside” references a nucleic acid subunit (DNA, RNA, or an analogue thereof) including a sugar group and a nitrogen-containing base. One skilled in the art would have the understanding that additional modification to the nucleoside may be appropriate (e.g., phosphorylation), and one skilled in the art has such knowledge. In typical usage, a phosphorylated nucleoside is a nucleotide.

A “nucleotide” or “nucleotide moiety” refers to a sub-unit of a nucleic acid (whether DNA or RNA or analogue thereof) that includes a phosphate group, a sugar group and a nitrogen-containing base, as well as analogs of such subunits.

A “nucleoside moiety” refers to a molecule having a sugar group and a nitrogen-containing base (as in a nucleoside) as a portion of a larger molecule, such as in a polynucleotide, oligonucleotide, or nucleoside phosphoramidite.

It will be appreciated that, as used herein, the terms “nucleoside” and “nucleotide” will include those moieties which contain not only the naturally occurring purine and pyrimidine bases, e.g., adenine (A), thymine (T), cytosine (C), guanine (G), or uracil (U), but also modified purine and pyrimidine bases and other heterocyclic bases which have been modified (these moieties are sometimes referred to herein, collectively, as “purine and pyrimidine bases and analogs thereof”). Such modifications include, e.g., diaminopurine and its derivatives, inosine and its derivatives, alkylated purines or pyrimidines, acylated purines or pyrimidines, thiolated purines or pyrimidines, and the like, or the addition of a protecting group such as acetyl, difluoroacetyl, trifluoroacetyl, isobutyryl, benzoyl, 9-fluorenylmethoxycarbonyl, phenoxyacetyl, dimethylformamidine, N,N-diphenyl carbamate, or the like. The purine or pyrimidine base may also be an analog of the foregoing; suitable analogs will be known to those skilled in the art and are described in the pertinent texts and literature. Common analogs include, but are not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine, and 2,6-diaminopurine.

As used herein the term “natural nucleoside” or “natural nucleotide” refers to naturally occurring purine and pyrimidine bases, e.g., adenine (A), thymine (T), cytosine (C), guanine (G), or uracil (U).

As used herein the term “nucleoside analogue” (NA) refers to a synthetic molecule that resembles a naturally occurring nucleoside, but that lacks a bond site needed to form an internucleotide bond to link the NA to an adjacent nucleotide. Thus, when incorporated into a forming polynucleotide, the polynucleotide will be terminated at the NA and thus prevented from forming the complete polynucleotide. Nucleoside analogs are also commonly referred to as nucleoside reverse transcriptase inhibitors, (NRTI), agents (e.g., AZT, 3TC, abacavir) that mimic one of the building blocks of genetic material and suppress retrovirus replication by interfering with the reverse transcriptase enzyme, causing premature termination of DNA copying.

A “fluorescent nucleoside analog” (fNA) refers to a NA that produces a detectable optical signal (e.g., fluorescence) outside the spectral range of regular cellular components, often referred to as “cellular autofluorescence”. More specifically, this definition includes, but is not limited to, nucleoside analogs whose spectroscopic property, specifically its maximum excitation wavelength, is above 300 nm.

An “internucleotide bond” refers to a chemical linkage between two nucleoside moieties, such as a phosphodiester linkage in nucleic acids found in nature or other linkages well known from the art of synthesis of nucleic acids and nucleic acid analogues. An internucleotide bond may include a phospho or phosphite group, and may include linkages where one or more oxygen atoms of the phospho or phosphite group are either modified with a substituent or replaced with another atom, e.g., a sulfur atom, or the nitrogen atom of a mono- or di-alkyl amino group.

The terms “nucleic acid” and “polynucleotide” are terms that generally refer to a string of at least, two base-sugar-phosphate combinations. As used herein, the terms include deoxyribonucleic acid (DNA) and ribonucleic acid (RNA) and generally refer to any polyribonucleotide or polydeoxyribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded, double-stranded, triple-stranded, or, more typically, a mixture of single- and double-stranded regions. The strands in such regions, particularly triple stranded regions, may be from the same molecule or from different molecules. The terms “nucleic acid sequence” and “oligonucleotide” also encompasses a nucleic acid and polynucleotide as defined above.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein.

The term also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotides” as that term is intended herein.

The term “prodrug” refers to an agent, including nucleic acids and proteins, which is converted into a biologically active form in vivo. For instance, NAs and fNAs of the present disclosure represent prodrugs, which are converted into active form in vivo by phosphorylation. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug, approaches to the improved delivery of peptide drugs. Curr. Pharm. Des., 5(4):265-87.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gin), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073, (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present invention.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution (including conservative and non-conservative substitution), or insertion, and wherein said alterations may occur at the amino- or carboxy-terminus positions of the reference polypeptide sequence or anywhere between those terminal positions, interspersed either individually among the amino acids in the reference sequence, or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated, mRNA and chemically aminoacylated suppressor tRNAs (Turcatti, et al., J. Biol. Chem., 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

As used herein “functional variant” refers to a variant of a protein or polypeptide (e.g., a circularly permuted protein, with or without additional sequence alterations) that can perform the same functions or activities as the original protein or polypeptide, although not necessarily at the same level (e.g., the variant may have enhanced, reduced or changed functionality, so long as it retains the basic function).

An “enzyme,” as used herein, is a polypeptide that acts as a catalyst, which facilitates and generally speeds the rate at which chemical reactions proceed but does not alter the direction or nature of the reaction.

As used herein, the term “2′-deoxyribonucleoside kinase” (dNK) refers to kinases that catalyze the phosphorylation of a 2′-deoxyribonucleoside to form the corresponding 2′-deoxyribonucleotide monophosphate. An exemplary dNK includes, but is not limited to, thymidine kinase. The term “2′-deoxyribonucleotide monophosphate kinase” (dNMPK) refers to kinases that catalyze the phosphorylation of a 2′-deoxyribonucleotide monophosphate to form the corresponding 2′-deoxyribonucleotide diphosphate (e.g., thymidylate kinase). The term “2′-deoxyribonucleotide diphosphate kinase” (dNDPK) refers to kinases that catalyze the phosphorylation of a 2′-deoxyribonucleotide diphosphate to form the corresponding 2′-deoxyribonucleotide triphosphate.

As used herein, the term “enhance,” “increase,” and/or “augment” generally refers to the act of improving a function or behavior relative to the natural, expected, or average. For example, a modified dNK that has increased activity with respect to a particular substrate over that of the corresponding native kinase has improved/increased activity (e.g., a faster rate of reaction, or binding/reacting with a greater number of substrates in the same amount of time) as compared to the activity of the corresponding native dNK.

The term “substantially similar” as used herein generally refers to a function, activity, or behavior that is close enough to the natural, expected, or average, so as to be considered, for all practical purposes, interchangeable. For instance, a protein with substantially similar activity would be one that has an activity level that would not be considered to be substantially more or less active than the native protein.

As used herein, the term “improvement” or “enhancement” generally refers to a change or alteration in a function or behavior of a protein, such as an enzyme, that in the applicable circumstances is considered to be desirable.

The term “substrate specificity” refers to the range of substrates that a polypeptide can act upon to produce a result. The term “broader substrate specificity” refers to a larger range of substrates that a polypeptide can act upon to produce a result, as compared to the native protein. The term “changed substrate specificity” refers to a different or altered range of substrates than a polypeptide can act upon to produce a result, as compared to the native protein. For instance, a modified kinase with broader substrate specificity, as compared to the respective native kinase, has the ability to phosphorylate a greater variety of substrates. A modified kinase with changed substrate specificity may have, for instance, increased activity with respect to a particular substrate and/or a preference for a particular substrate, as compared to the activity and/or preference of the native kinase for the same substrate. As used herein “modified substrate specificity” may include broader and/or changed substrate specificity. Thus, a modified kinase with modified substrate specificity could have broader substrate specificity, changed substrate specificity or both.

As used herein, a “detectable fluorescent signal” or a “detectably effective amount” of a compound refers to a signal that can be sufficiently distinguished by methods known to those of skill in the art from background signal, such as natural autofluorescence. For instance, in embodiments a detectable fluorescent signal for an fNA of the present disclosure would have fluorescence at over 300 nm, which is detectable over that of any background autofluorescence.

Having defined some of the terms herein, various embodiments of the disclosure will be described in additional detail below.

### DESCRIPTION

Briefly described, embodiments of the present disclosure include novel fluorescent nucleoside analogs (fNAs); methods of making the novel fNAs; methods of using fNAs to detect phosphorylation of fNAs in vivo; and methods of using fNAs to identify dNKs (e.g., naturally occurring as well as modified and/or novel kinases) with increased activity for phosphorylation of fNAs and/or changed substrate specificity for fNAs. These embodiments will be described in greater detail in the discussion and examples below.

Fluorescent Nucleoside Analogs (fNAs)

The ability to detect nucleosides and nucleoside analogues with high sensitivity in complex mixtures such as a cell's cytoplasm would greatly benefit studies of cellular uptake and metabolism. While the nucleobases of natural nucleosides do possess intrinsic fluorescence properties at physiological conditions, direct measurements are impractical due to the compounds' low quantum yields and overlapping absorption maxima with aromatic amino acids in proteins and small-molecule metabolites such as flavines and NADH. Due to such cellular autofluorescence, fluorescent substrates or reporters with absorption maxima of >300 nm are highly sought after to minimize background and improve signal-to-noise ratios.

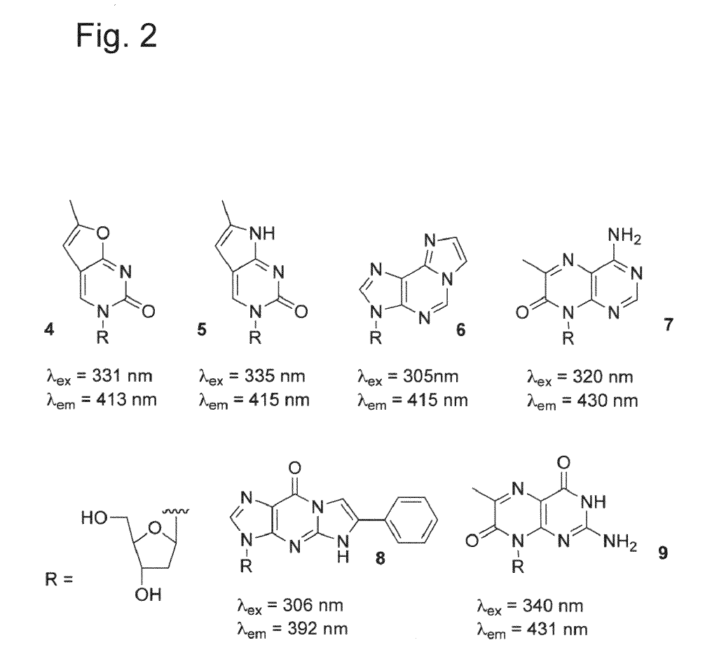

Among the first modified nucleosides with improved fluorescent properties were the etheno-derivatives of adenosine (FIG. 2, compound 3) and cytidine. A tricyclic guanine derivative (FIG. 2, compound 5) has also been described. While these fluorescent analogs are accepted by a number of nucleotide-utilizing proteins, numerous additional fluorophors have since been synthesized to address specific biological questions. More importantly, the preparation of new fNAs is being driven by the need for fluorescent probes with improved spectral properties but that still closely mimic the natural nucleobases in size and hydrogen-bonding patterns. Among these second and third-generation compounds, the furano and pyrrolo-pyrimidines (FIG. 2, compounds 1 and 2) and the pterines (FIG. 2, compounds 4 and 6) are of particular interest for methods of the present disclosure for monitoring in vitro and in vivo phosphorylation of NA prodrugs. These fNAs show close structural similarities with natural nucleobases, and their red-shifted excitation and emission wavelengths further reduce the interference of cellular autofluorescence in experiments.

### Pterines as Purine Analogs

The pterine derivatives, FIG. 2, compounds 4 (4-amino-6-methyl-8-(2′-deoxy-β-D-ribofuranosyl)-7(8H)-pteridone) and 6 (2-amino-6-methyl-8-(2′-deoxy-β-D-ribofuranosyl) pteridine-4,7(3H,8H)-dione) are synthetically accessible in three steps via direct coupling of the corresponding pterine moieties to ribofuranosyl chloride. When incorporated into oligonucleotides, fNAs 4 and 6 form hydrogen bonding interactions similar to the natural nucleotides and show minimal interference with the secondary structure of the macromolecule as demonstrated in DNA melting studies. Enzymatic studies have not been reported in the literature.

### Pyrimidine Analogs

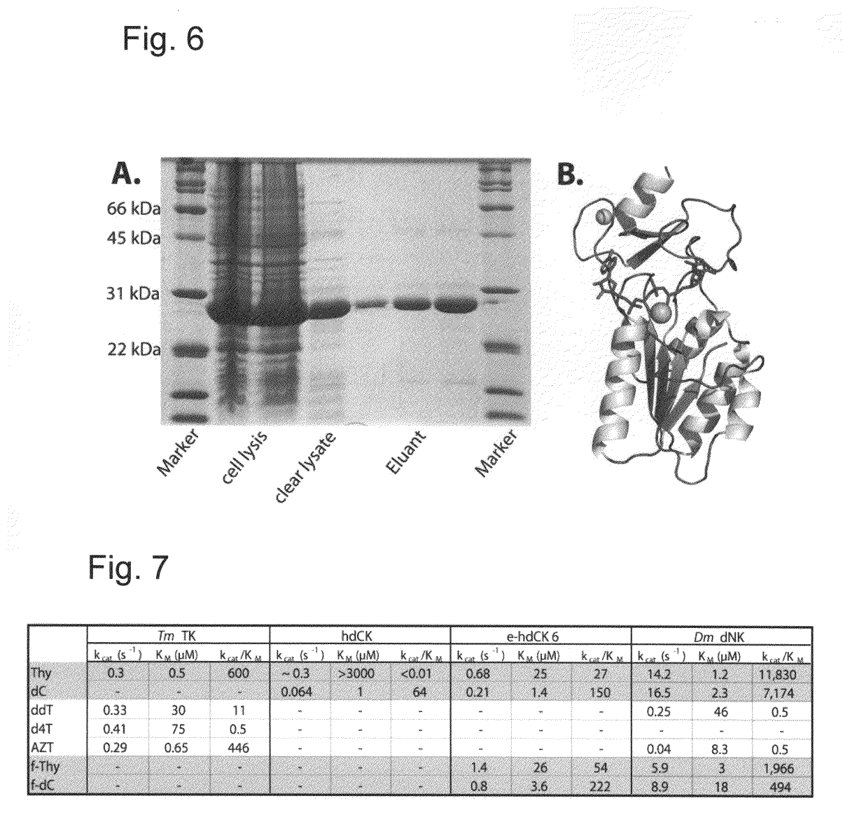

The fluorescent thymidine analog 6-methyl-3-(β-D-2′-deoxyribofuranosyl)furano-[2,3-d]pyrimidin-2-one (FIG. 2, compound 1) and the cytidine analog 6-methyl-3-(6-D-2′-deoxyribofuranosyl)-3H-pyrrolo[2,3-d]pyrimidin-2-one (FIG. 2, compound 2) have been used to study, the structural and functional properties of nucleic acids and more recently nucleoside membrane transport proteins. When incorporated in synthetic oligonucleotides, 2 forms hydrogen-bonding interactions equivalent to its natural counterpart and causes no significant structural disturbance. Similar “native-like” behavior is also exhibited by 1 and 2 in enzymatic assays, and both compounds show low cytotoxicity in vivo. As outlined in FIG. 5A, fNA 1 is synthetically accessible from readily available 2′-deoxy-uridine (17) in three steps, using palladium-catalyzed cross coupling of an alkyne to the 5-halogenated nucleoside derivative 18, followed by cyclization to form the fluorescent furano-pyrimidine nucleoside 1 (as described in Robins, M. J. and Barr, P. J. (1983) Nucleic-Acid Related-Compounds 0.39. Efficient Conversion of 5-Iodo to 5-Alkynyl and Derived 5-Substituted Uracil Bases and Nucleosides. Journal of Organic Chemistry, 48, 1854-1862, which is incorporated herein by reference). Subsequent treatment of compound 1 in methanolic ammonia allows the direct conversion to compound 2 (FIG. 5B) (as described in Woo, J., Meyer, R. B., Jr. and Gamper, H. B. (1996) G/C-modified oligodeoxynucleotides with selective complementarity: synthesis and hybridization properties. Nucleic Acids Res, 24, 2470-2475, which is incorporated herein by reference).

Novel Fluorescent Nucleoside Analogs with Ribose Modifications

The present disclosure provides novel fluorescent nucleoside analogs (fNAs) having modifications to the sugar moiety (e.g., the 2′-deoxyribose or ribose moiety).

Very few versions of fluorescent nucleoside analogs with modified sugar moieties (e.g., sugars other than ribose and 2′-deoxyribose) have been reported. Searching for acyclovir and ganciclovir derivatives with increased antiviral activity, a range of tricyclic guanine NA derivatives with substitutions in the appended ring, such as the highly fluorescent 6-phenyl analog 5 (FIG. 2) have been developed. In vivo studies of these fNAs in HSV-infected mammalian cells, conducted in parallel with the synthetic efforts, suggested the efficient phosphorylation of several of the analogs by deoxynucleoside kinases. More recent in vitro experiments, however, show that turnover of fNA 5 by the HSV-kinase is 10 to 100-fold lower in comparison to ganciclovir.

Focusing exclusively on the pharmacological properties of these modified nucleosides, their potential use in connection with studies of the cellular metabolism and, in particular, for the identification of dNKs is completely unexplored. Arabinosyl derivatives of the furano-pyrimidine have also been prepared. The evaluation of this fluorescent araT against varicella zoster virus (VZV) however revealed significantly reduced biological activity of the furano product compared to araT. Similar substrate limitations for type-II dNK have also been observed, as discussed in greater detail in the examples below. Without being bound by theory, it is believed that these observations are linked to the furano-pyrimidine rather than the ribose modifications.

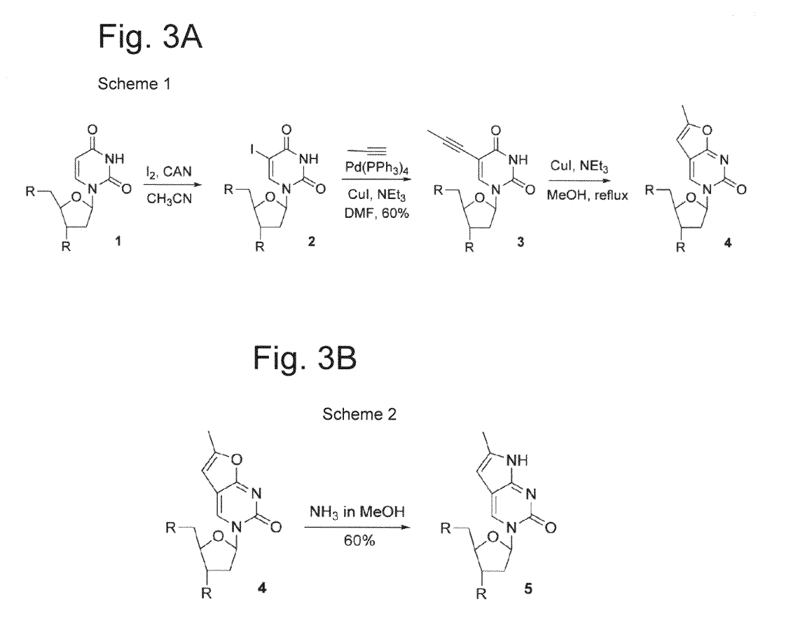

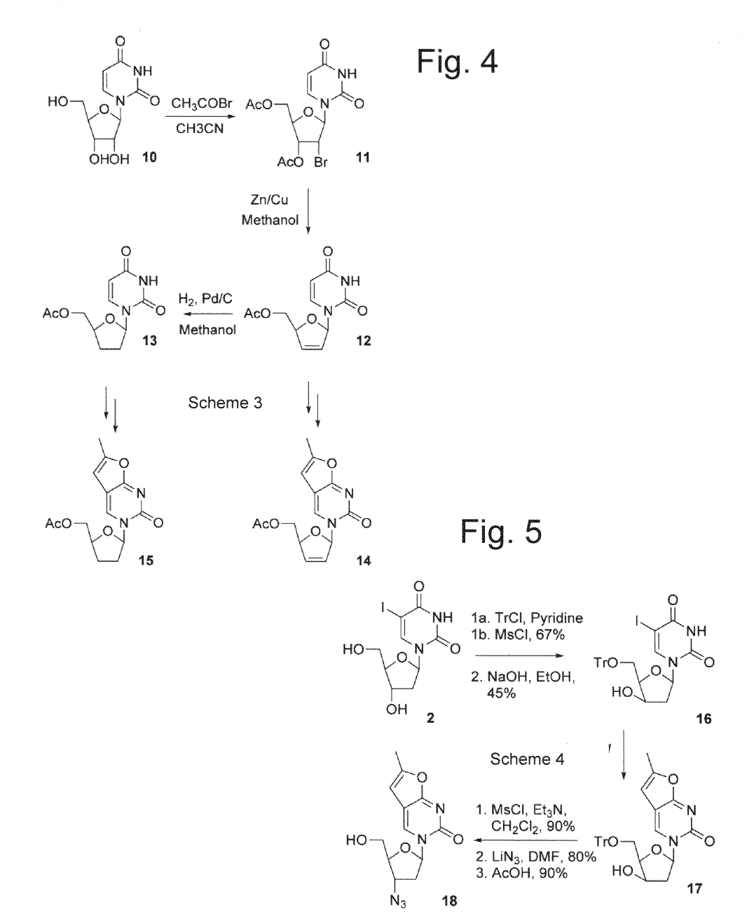

Embodiments of the present disclosure provide novel fluorescent nucleoside analogs including a fluorescent analog of a nucleic acid subunit (e.g., a purine or pyrimidine base) and a modified sugar moiety. For instance, in some exemplary embodiments, as shown in FIGS. 2, 3 and 4, a series of exemplary fNAs can be prepared by combining an individual sugar derivative (FIG. 4) with a fluorescent heterocycle (FIGS. 2 and 3). Exemplary novel fNAs include a modified sugar moiety, including, but not limited to, compounds 10-16 of FIG. 4, and a fluorescent nucleobase analog, including but not limited to, compounds 1-9 of FIGS. 2 and 3. It should be noted that while FIGS. 2 and 3 show the sugar moiety (R group for FIG. 2) as being deoxy ribose, the sugar moiety can be any modified sugar derivative, such as, but not limited to, those illustrated in FIG. 4 (compounds 10-16). Likewise, for compounds 10-16 of FIG. 4, while the nucleobase (R group) is shown as that of compound 1, the nucleobase can be any fluorescent nucleobase (such as a fluorescent purine or pyrimidine base or analog thereof) such as, but not limited to, those fluorescent nucleobases illustrated in FIGS. 2 and 3 (compounds 1-9).

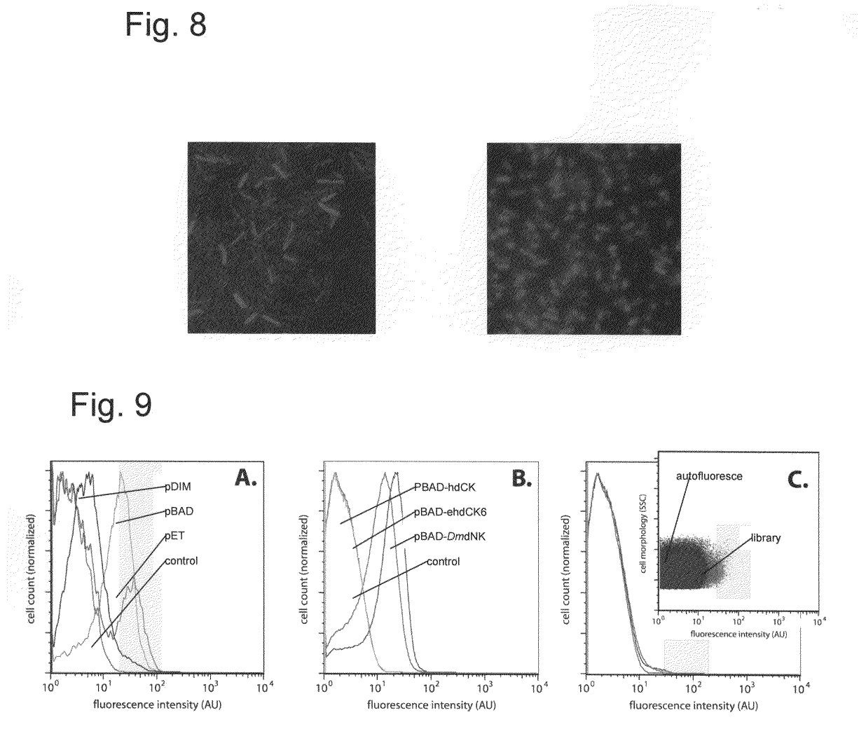

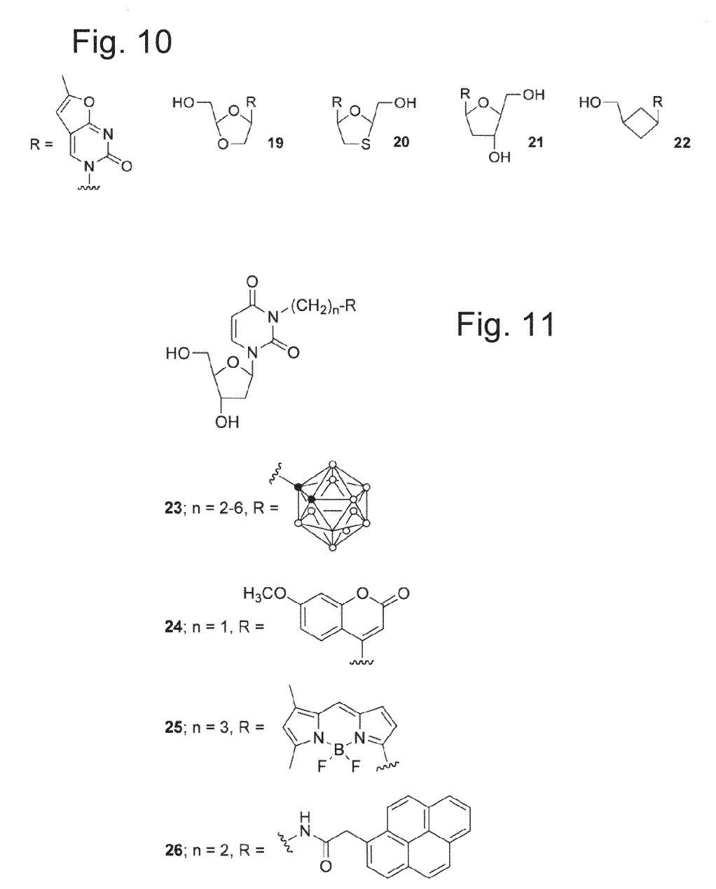

The present disclosure also provides methods of making the novel fNAs of the present disclosure. Additional detail regarding, exemplary fNAs of the present disclosure and methods of making some exemplary fNAs of the present disclosure are provided in FIGS. 5-7 and in the discussion and examples below. For instance, novel variants of compounds 1 and 2 can be prepared with the synthesis embodiments of FIGS. 6 and 7. Two exemplary embodiments include fNA 3-(4-azido-5-(hydroxymethyl)-tetrahydrofuran-2-yl)-6-methylfuro[2,3-d]pyrimidin-2(3H)-one (16) (a variant of compound 1 that is a combination of sugar derivative from FIG. 4 and fluorescent nucleobase from FIG. 2), and fNA 3-(5-(hydroxymethyl)-tetrahydrofuran-2-yl)-6-methyl-3H-pyrrolo[2,3-d]pyrimidin-2(7H)-one (25) (a variant of compound 1 that is a combination of sugar derivative 14 from FIG. 4 and fluorescent nucleobase 2 from FIG. 2) can be prepared as illustrated in FIGS. 6 and 7, respectively and described in detail in Example 1.)

In addition to the novel furano and pyrrolo-nucleosides with ribose moiety modifications, described in the examples below and illustrated in FIGS. 6 and 7, the present disclosure provides thymine and cytosine derivatives, and synthetic strategies for preparing additional nucleoside analogs with unnatural ribose moieties. Exemplary variants include the dioxilanes (FIG. 4, compound 10), L-3′-thia-nucleosides (FIG. 4, compound 11), L-nucleosides (FIG. 4, compound 12), and cyclobutanes (FIG. 4, compound 13). Embodiments of the present disclosure include a compete library of fluorophors with the above-described sugar modifications. The selection of these sugar derivates is based on their medicinal relevance. While all compounds could benefit from more efficient intracellular activation, L-nucleosides and their thia derivatives have already proven highly selective for antiviral treatment and dioxilanes and cyclobutanes hold promise for the next generation of antivirals.

It is believed that all four derivatives (10-13) can be prepared by the same procedure as outlined in FIG. 6, creating the furano-pyrimidine system in the last step of the nucleoside analog synthesis. Alternatively, the coupling of the finished nucleobase to ribofuranosyl chloride as discussed herein for the pterine NAs may be performed. These compounds were subsequently tested in vitro and in vivo (as described in Examples 2 and 3), evaluating in particular their phosphorylation by deoxynucleoside kinases.

Furthermore, the present disclosure includes modified fNA's beyond the pyrimidine derivatives and synthetic protocols for the preparation of adenine and guanosine nucleoside analogs with modified ribose moieties. Exemplary modified fNAs include pterine derivatives 4 and 6 (FIG. 2), with modified sugar moieties. Pteridine-based NAs are attractive as they are synthetically easily accessible via direct coupling of the pterine moiety to the ribose, and as they possess favorable spectroscopic properties compared to previously reported fNAs.

As little data about the behavior of these fNAs with enzymes is known in the literature, the present disclosure includes 2′-deoxyribose versions of both the adenosine and guanosine analogs. Validation of the suitability of these compounds for FACS-based in vivo kinase assays, described below, sets the stage for the synthesis of various pterine-based fNAs with modified ribose portions and could also prove invaluable as an enzymatic route for preparing 5′-phosphorylated fNAs with application in nucleotide-protein interaction studies.

Using fNAs to Improve Cellular Metabolism of NAs

In light of the strength and versatility of these compounds, the application of fluorescent nucleoside analogs offers new intriguing opportunities for the investigation of cellular uptake and metabolism of nucleosidic prodrugs. Embodiments of the present disclosure also provide for the use of fNAs of the present disclosure to detect phosphorylation of fNAs in vivo and to evaluate the efficiency of phosphorylation of various fNAs. The present disclosure also provides for the use of fNAs of the present disclosure as substrates in high-throughput screening assays for identifying dNKs that can utilize selected NAs. Briefly described, methods of detecting in vivo phosphorylation of NAs includes contacting a cell with one or more fNAs and detecting the change in fluorescence intensity inside the cell. An increase in fluorescence intensity in the cell indicates the accumulation of fNAs inside the cell thereby indicating the phosphorylation of the fNAs, since once phosphorylated, the fNAs can no longer pass freely in and out of the cell. The change in fluorescence intensity can be detected and/or quantiated by use of methods such as, but not limited to, fluorescence microscopy. In embodiments, the methods of the present disclosure also include detecting the distribution of the fNAs inside the cell. In some embodiments, a population of cells is contacted with the solution of fNAs, and fluorescence-activated cell sorting (FACS) is used to detect and isolate cells with increased fluorescence. Exemplary embodiments of the use of fNAs to detect in vitro phosphorylation are described in the embodiments below.

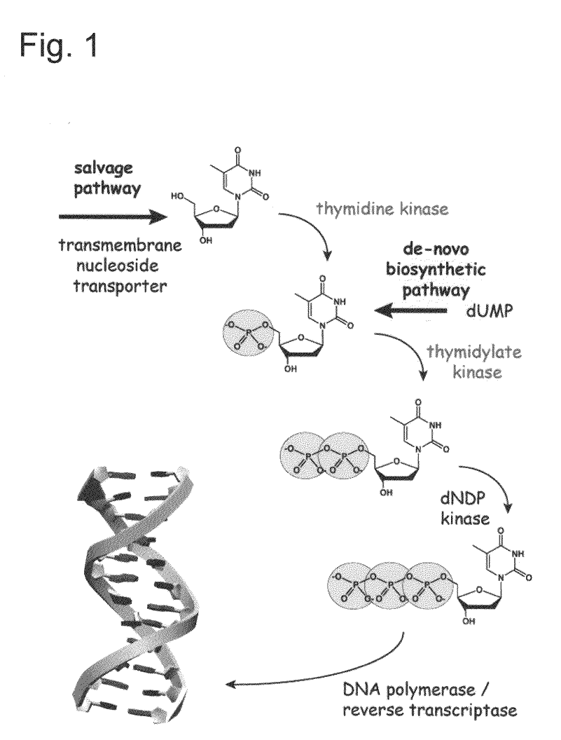

As discussed above, the phosphorylation of NAs by cellular kinases often represents the rate-determining step in the bioavailability of a prodrug. Therefore, determining the in vivo efficiency of phosphorylation of a particular NA prodrug is important in evaluating the effectiveness of that prodrug. To address this need, embodiments of the present disclosure provide methods of detecting and analyzing in vivo phosphorylation of fNAs. In embodiments, the phosphorylative activation of fNAs of the present disclosure is monitored in vivo by detecting an increase in fluorescence intensity in a cell, indicating accumulation of phosphorylated fNA compounds inside the cell. For instance, in an exemplary embodiment, the initial phosphorylation of an fNA from the deoxynucleoside to its corresponding 5′-monophosphate derivative (see FIG. 1) by a 2′-deoxynucleoside kinase (dNKs) is detected and/or quantiated by detecting/quantiating an increase in fluorescence intensity as the fNA-monophosphate accumulates inside a cell, as the monophosphate version of the fNA is no longer able to diffuse in and out of the cell.

The shortcomings of the cellular phosphorylation cascade with respect to NAs has led to exploration of other approaches, such as a combined therapy with a prodrug and an exogenous broad-specificity dNK such as thymidine kinase from herpes simplex virus (HSV-TK). Co-administered through a retroviral gene delivery system, the broad-specificity kinase carries out the initial phosphorylation of the NA prodrugs. The method has proven effective in the treatment of malignant tumors in combination with ganciclovir, and has recently been successfully tested in conjunction with AZT on HIV-infected lymphoid and monoblastoid cells. However, while experiments with HSV-TK show overall increased host sensitivity for NAs, kinetic data suggest only sub-optimal performance of the wild-type enzyme, which is largely contributed by HSV-TK's poor substrate binding affinity. Over the last decade, several groups have therefore sought to improve the prodrug-activation approach by engineering HSV-TK, as well as other dNKs and dNMPKs by rational redesign and directed evolution.

### Engineering Deoxynucleoside Kinases

Taking advantage of powerful new combinatorial techniques such as directed evolution to engineer proteins to specification, various laboratories have been searching for more efficient prodrug-activating kinases. Starting with the wild-type sequence of HSV-TK, approaches including site-directed, cassette, and random mutagenesis have led to the identification of several TK derivatives that display ten to hundred-fold improvements in substrate specificity. However, associated with the gain in specificity is a significant reduction of enzyme activity by two to three orders of magnitude in all characterized mutants. More promising dNK engineering efforts, resulting in a gain of specificity while maintaining the overall activity, were achieved by protein fragment substitution of entire secondary structures and domains. Using DNA shuffling, the random recombination of fragments of the closely related HSV-TK type 1 and 2 generated hybrid dNKs with up to 100-fold higher specificity to AZT while retaining parental activity. These promising results certainly suggest that an extension of the shuffling experiments involving a broader range of parental sequences could lead to the identification of more diverse and highly active hybrid enzymes.

Such desires were reinforced by the identification of a new, promising dNK in Drosophila melanogaster (DmdNK). The wild-type enzyme possesses broad substrate specificity, phosphorylating all natural deoxynucleosides, as well as a variety of analogues with high efficiency. Initial engineering attempts have focused on site-directed and random mutagenesis, producing, as in the case of HSV-TK, mutant enzymes with improved substrate specificities but a significant reduction in overall enzyme activity. Most recently chimeragenesis, in addition to random mutagenesis and DNA shuffling, has been applied as a method for creating sequence diversity. However, in addition to ongoing efforts for creating larger, more diverse libraries of dNKs from various promising parents, kinase engineers are facing a fundamental problem with the large, diverse combinatorial libraries. As for all successful directed evolution experiments, generating large libraries is optional, but being able to identify and recover the desired progeny is mandatory.

Identifying Modified Kinases with Desired Catalytic Activities

Current kinase engineering efforts have critical limitations. For the last 20 years, the field has relied on two techniques for library analysis: a) in vivo selection, using genetic complementation of the auxotrophic E. coli strain KY895, or b) in vivo screening on replica plates, testing for NA phosphorylation via negative selection in connection with cytotoxicity. Both methods have serious limitations. The auxotrophic selection only tests for thymidine kinase activity, resulting in a bias towards enzymes with (at best) broad substrate specificity. Replica plating is very laborious and ultimately relies on the cytotoxicity of the phosphorylated NA, a criteria that, in testing, applied to AZT but none of the other NAs listed in this proposal. In light of these biases and functional limitations of the existing assay systems, it is believed that the failure to identify highly active dNKs for individual NAs can largely be attributed to the inadequate selection or screening techniques.

Methods of the present disclosure employing fluorescent nucleosides and nucleoside analogs now offer a new method for identifying enzymes for the efficient phosphorylation of various NAs. In embodiments, the present disclosure also provides methods of screening for and identifying kinases (both type I and type II kinases) that have increased activity for phosphorylation of NAs. In embodiments, methods provide for identifying novel dNKs with modified substrate specificity, in particular with changed substrate specificity with respect to NAs. For instance, novel kinases identified by methods of the present disclosure may have increased substrate specificity for NAs as compared to natural nucleosides. Methods of screening for kinases with increased substrate specificity/activity for NAs include using fNAs in combination with assays using cells engineered to overexpress kinases of interest. In other embodiments, populations of cells are screened using fluorescence-activated cell sorting (FACS) to identify novel kinases capable of phosphorylating NAs. These methods can be used with a variety of host cells for in vitro or in vivo screening. Exemplary host cells include, but are not limited to, bacterial cells, yeast and/or other fungal cell systems, as well as to mammalian cell lines, in particular certain cancer cell lines. For instance, in order to test for kinases capable of phosphorylating NAs useful in the treatment of a particular cancer, cancer cell lines can be ideally used in the screening methods of the present disclosure to optimize results.

In exemplary embodiments of methods of screening for kinases capable of phosphorylating an NA of interest, an fNA corresponding to the NA of interest is provided. Then a cell is provided that over-expresses a kinase of interest. When the cell is contacted with a composition of the fNA, an increase in fluorescence inside the cell indicates accumulation of the fNA in the cell, indicating phosphorylation. In other methods, a population of cells can be screened, with the population of cells including subpopulations where each subpopulation expresses or over-expresses different kinases of interest. Then, the population of cells is contacted with a composition of fNAs and screened (such as by FACS) to identify cells having an increase in fluorescence indicating phosphorylation of the fNA. The cells used can include a variety of host cells including, but not limited to, bacterial, fungal, and animal cells, including mammalian cells. The cells can be engineered to express and/or over-express one or more kinases of interest. Exemplary screening methods are described in more detail in the Examples below.

Following the chemical synthesis of a series of fNAs, as in the examples below, results indicate that the fNAs of the present disclosure allow for the direct, positive detection of phosphorylative activation of NAs by dNKs. Conceptually, when bacteria that express individual enzyme variants are incubated with a particular fNA, the fluorophor can efficiently be moved in and out the cell. In contrast, the product of the enzymatic reaction, fNA-monophosphate, carries a negative charge, preventing it from leaving the cellular compartment. Consequently, the product accumulates inside the cell that carries a functional kinase, leading to increase fluorescence, which can be detected by fluorescence microscopy.

Additionally, fluorescent host organisms can be identified and isolated by fluorescence-activated cell sorting (FACS). At screening rates of up to 200,000 cells per hour, FACS can facilitate the analysis of extensive protein engineering libraries. Host cells with fluorescence intensity above the control background can be collected for further analysis. As for engineered dNKs, the post-screening analysis involves the sequencing of the plasmid-encoded exogenous dNK, followed by its overexpression, purification, and in vitro characterization. The FACS-based assay gives additional flexibility in that the screening protocol can be iterative under increasingly stringent selection conditions such as shorter incubation times and lower concentration of the fluorescent probe in the reaction mixture.

Furthermore, the system allows for consecutive selection cycles where the fluorescent probe is alternated between a particular NA and a natural nucleoside. Collecting cells that show high fluorescence with the NA but reduced intensity with the natural substrate will result in the enrichment for kinase candidates with new, changed substrate specificity as opposed to merely broader activity. The examples below describe some exemplary embodiments of methods of screening nucleoside kinases for NA activation.

Additional Applications of fNAs

In addition, the fNAs of the present disclosure and methods of use of the fNAs could prove valuable for in vitro and in vivo studies of specific nucleoside analog-protein interactions (e.g. G-proteins and phosphatases).

It should be emphasized that the above-described, embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

### EXAMPLES

### Example 1

### Synthesis of Fluorescent Nucleoside Analogs

Extending upon the established synthesis of the 2′-deoxy ribose nucleoside analogs 1 and 2 (FIG. 2), a first set of novel furano and pyrrolo-nucleosides with modification in the ribose portion was prepared. Fluorescent analogues of the prodrugs ddT, ddC, and d4T were prepared as outlined in FIG. 6. Briefly, reduction of uridine (21) led to 2′,3′-didehydro-2′,3′-dideoxy (23) and 2′,3′-dideoxy (24) uridine analogs, which subsequently were converted to the corresponding furano-pyrimidines 14 and 15. Both compounds can be prepared in large quantities and good yields by this synthesis. Ammonia treatment of compounds 14 and 15 yielded the corresponding pyrrolo-cytidine derivatives.

Separately, the fluorescent AZT analog 16 was prepared. Synthesis of the 3′-azido sugar derivative from its 5-halogenated analog, following the strategy outlined in FIG. 5A was difficult due to the reductive amination of the azido group in the cross-coupling reaction step. Instead, the azido group was introduced stereoselectively after modification of the nucleobase was completed as shown in FIG. 7. Preparation of the 2,3′-anhydro intermediate via the furano-threo-derivative 27, followed by nucleophilic displacement with lithium azide produced the fluorescent AZT analog 16 in high yields.

The spectral properties of nucleoside analogues 14, 15, and 16 were investigated by fluorescence spectroscopy. The three compounds showed virtually identical excitation and emission wavelengths as compound 1. Interestingly, the quantum yields for 1, 14, and 16 are very similar, while the fluorescence, intensity of 15 increased approximately three-fold. It is speculated that the spectral improvement in 15 is linked to its extended fluorescence lifetime, resulting from increased conformational rigidity of its ribose moiety.

### Synthesis of 3-(4-azido-5-(hydroxymethyl)-tetrahydrofuran-2-yl)-6-methylfuro[2,3-d]pyrimidin-2(3H)-one (16) (FIG. 7)

1-(4-hydroxy-5-(trityloxymethyl)-tetrahydrofuran-2-yl)-pyrimidine-2,4(1H,3H)-dione (27)—Treatment of 5-iodo-2′-deoxyuridine (18) with trityl chloride in pyridine followed by addition of methanesulphonyl chloride provided protected compound. Following the published procedure (described in McGuigan C., Carangio A., Snoeck R., Andrei G., De Clercq E., Balzarini J. (2004) Synthesis and Antiviral Evaluation of Some 3′-Fluoro Bicyclic Nucleoside Analogues; Nucleosides, Nucleotidesand Nucleic Acids 23, 1-5, incorporated herein by reference), the conformation of the 3′-group was readily inverted to give the xylo compound 27 using refluxing ethanic sodium hydroxide, which was converted to fluorescent compound 28 via Sonogashira reaction condition followed by cyclization in triethylamine/methanol with copper(I) iodide. Treatment of 28 with methanesulphonyl chloride, and then replacement of the methanesulphonyloxy group using lithium azide in DMF gave intermediate, which was deprotected in 80% aqueous acetic acid to provide the 3′-azido fluorescent nucleoside 16.

1-(4-hydroxy-5-(trityloxymethyl)-tetrahydrofuran-2-yl)-5-(prop-1-ynyl)pyrimidine-2,4(1H,3H)-dione (27a)—A stirred solution of compound 27 (506 mg, 0.85 mmol) in anhydrous DMF (10 ml), in a three neck flask was deoxygenated with argon for 1.5 h. Catalyst tetrakis(triphenylphosphine)palladium (100 mg, 0.085 mmol) and copper(I) iodide (30 mg, 0.17 mmol) were added. Argon was removed by vacuum, and anhydrous triethylamine was injected and the flask was filled up with propyne (1.5 ml, 25.5 mmol). The reaction mixture was protected from light and stirred for 22 h., then triphenylphosphine on polystyrene was added to deactivate catalyst. The mixture was stirred in argon for 3 h. The resin was filtered and washed with methanol, and the combined filtrate was evaporated to dryness. The crude product was purified by flash column chromatography (initial eluent: ethyl acetate/hexane (1:1), followed by: ethyl acetate). The appropriate fractions were combined, and the solvent was removed in vacuo to give product (260 mg, 62%). IR (cm⁻¹) 3440 (br), 3195 (br), 3056 (br), 1691, 1446, 1286, 1061, 706; ¹H NMR (400 MHz, CDCl₃) δ 1.85 (s, 3H), 2.26 (d, 1H, J=14.4), 2.48-2.57 (m, 1H), 3.39 (s, 1H), 3.45-3.71 (m, 2H), 4.10 (m, 1H), 4.36 (s, 1H), 6.13 (d, 1H, J=6.6), 7.25-7.35 (m, 9H), 7.48 (d, 6H, J=7.5), 7.99 (s, 1H), 9.69 (s, 1H); ¹³C NMR (100 MHz CDCl₃) δ 4.9, 41.5, 62.6, 70.7, 70.8, 84.3, 86.5, 87.6, 90.5, 100.0, 127.5, 128.3, 128.8, 143.6, 149.9, 163.0, 179.0; M+H⁺, C₃₁H₂₉N₂O₅ 509.2070.

3-(4-hydroxy-5-(trityloxymethyl)-tetrahydrofuran-2-yl)-6-methylfuro[2,3-d]pyrimidin-2 (3H)-one (28)—To a stirred solution of compound 27a (260 mg, 0.52 mmol) in methanol (70 ml) and triethylamine (30 ml) (7:3) was added copper(I) iodide (19 mg, 0.10 mmol). The mixture was reflux for 4 h. The solvent was removed in vacuo, and the crude product was purified by flash chromatography (initial eluent: ethyl acetate, followed by ethyl acetate/methanol (9:1). The combined fractions were combined and the solvent was removed in vacuo to give the pure product 28 (180 mg, 70%). IR 3342 (br), 3060 (br), 1674, 1572, 1446, 1074, 706; ¹H NMR (400 MHz, CDCl₃) δ 2.25 (s, 3H), 2.45-2.57 (m, 2H), 3.52 (dd, 1H, J=3.2, 10.4), 3.79 (dd, 1H, J=6.8, 10.8), 4.279 (m, 1H), 4.39 (m, 1H), 5.91 (d, 1H, J=1.2), 6.12 (d, 1H, J=5.6), 7.23-7.32 (m, 9H), 7.52 (d, 6H, J=7.2), 8.35 (s, 1H); ¹³C NMR (100 MHz, CDCl₃) δ 14.3, 42.1, 63.2, 70.9, 85.8, 87.4, 89.4, 100.2, 107.0, 127.4, 128.2, 128.8, 129.0, 137.1, 143.9, 155.0, 155.0, 171.6; M+H⁺, C₃₁H₂₉O₅N₂ 509.2068.

5-(6-methyl-2-oxofuro[2,3-d]pyrimidin-3(2H)-yl)-2-(trityloxymethyl)-tetrahydrofuran-3-yl methanesulfonate (28a)—To an ice-cooled solution of compound 28 (100 mg, 0.20 mmol) and triethylamine (0.057 ml, 0.40 mmol) in dichloromethane (15 ml) was added methanesulfonyl chloride (0.030 ml, 0.40 mmol) dropwise. The reaction mixture was kept at room temperature for 1 h. The solvent was removed in vacuo, and the crude product was purified by flash chromatography (initial eluent: ethyl acetate, followed by ethyl acetate/methanol (9:1). The combined fractions were combined, and the solvent was removed in vacuo to give the crude product as foamy solid (100 mg, 87%). IR (cm⁻¹) 3620 (br), 3520 (br), 1670, 1642, 1576, 1172; ¹H NMR (400 MHz, CDCl₃) δ 2.31 (s, 1H), 2.58 (d, 1H, J=16), 2.91 (m, 1H), 3.45 (dd, 1H, J=12.8, 6.4), 3.69 (m, 1H, J=10.4, 6.4), 4.41 (m, 1H), 5.21 (t, 1H, J=3.6, 4.0), 5.97 (d, 1H, J=1.2), 6.23 (m, 1H), 7.29 (m, 9H), 7.48 (m, 6H), 8.07 (s, 1H); ¹³C NMR (100 MHz, CDCl₃) δ 14.4, 38.6, 40.8, 61.9, 79.6, 83.2, 87.7, 87.8, 99.9, 107.8, 127.7, 128.3, 128.9, 134.5, 143.5, 154.9, 156.1, 172.2; M+H⁺C₃₂H₃₁O₇N₂S₁ 587.1847.

3-(4-azido-5-(hydroxymethyl)-tetrahydrofuran-2-yl)-6-methylfuro[2,3-d]pyrimidin-2(3H)-one (16)—Compound 28a (80 mg, 0.14 mmol) and LiN₃ (12 mg, 0.20 mmol) were suspended in DMF (5 ml) and the mixture was heated to 110° C. for 2.5 h. The organic homogeneous mixture was poured into water and ethyl acetate was added. The aqueous layer was extracted with ethyl acetate and the organic extract was washed with water, then with brine. After drying (Na₂SO₄), the solvent was evaporated in vacuo to give product as a foamy solid (58 mg, 80%). A solution of the foamy solid (40 mg, 0.075 mmol) in aqueous acetic acid (80%, 20 ml) was heated to 90° C. for 15 mins. After cooling the solvent was removed under reduced pressure, and the residue was purified by flash chromatography with ethyl acetate (1.5% methanol) as eluant. Evaporation of the appropriate fractions afforded the title compound (13 mg, 60%), which was crystallized from ethyl acetate. IR (cm⁻¹) 3469 (br), 2839, 1667, 1638, 1573, 1482; ¹H NMR (400 MHz, CDCl₃) δ 2.27 (d, 3H, J=7.2), 2.30-2.36 (m, 1H), 2.57-2.64 (m, 1H), 3.29 (brs, 1H), 3.71-3.75 (m, 1H), 3.90-3.95 (m, 2H), 4.18 (dd, 1H, J=6.8, 13.2), 6.10-6.14 (m, 1H), 8.65 (s, 1H); ¹³C NMR (100 MHz, CDCl₃) δ 14.1, 39.2 58.6, 60.5, 85.7, 88.0, 100.1, 108.4, 136.2, 155.2, 156.2, 171.9; M+H⁺, C₁₂H₁₄O₄N₅ 292.1036.

### Synthesis of 3-(5-(hydroxymethyl)-tetrahydrofuran-2-yl)-6-methyl-3H-pyrrolo[2,3-d]pyrimidin-2(7H)-one (FIG. 6)

Compound 24 was converted into the 5-iodo derivative, which in turn was transformed into the 5-alkylnyl derivative. Following the same procedures, this compound was readily converted into the fluorescent nucleoside 14 using a two-step Pd- and Cu-catalyzed process. The ammonia exchange method of Woo et al. ((1996) G/C-modified oligodeoxynucleotides with selective complementarity: synthesis and hybridization properties, Nucleic Acids Res., 24, 2470-2474, which is hereby incorporated by reference) was employed to give pyrrolo compound 25.

(5-(5-iodo-2,4-dioxo-3,4-dihydropyrimidin-1(2H)-yl)-tetrahydrofuran-2-yl)methyl acetate—A mixture of compound 24 (as described in Ciurea, A.; Fossey, C.; Benzaria, S.; Gavriliu, D.; Delbederi, Z.; Lelong, B.; Laduree, D.; Aubertin, A. Kim, A. (2001) Synthesis of 5-alkenylated D4T Analogues via the Pd-catalyzed Cross-coupling Reaction. Nucleosides, Nucleotides & Nucleic Acids, 20, 1655-1670, incorporated by reference herein) (1.27 g, 0.5 mmol), iodine (76 mg, 0.3 mmol), CAN (137 mg, 0.25 mmol), and MeCN (8 ml) was stirred at 80° C. for 1 h. Reaction progress was monitored by TLC. Solvent was evaporated, and the residue was partitioned between a cold solution of EtOAc (20 ml), saturated NaCl/H₂O (10 ml), and 5% NaHSO₃/H₂O (5 ml×2), dried (MgSO₄) and evaporated. The crude 5-iodo products were purified by flash chromatography to give 1.75 g product in 92% yield. IR (cm⁻¹) 3230 (br), 3080 (br), 1736, 1701, 1683; ¹H NMR (CDCl₃) δ 1.78-1.88 (m, 1H), 1.98-2.04 (m, 1H), 2.09-2.19 (m, 1H), 2.41-2.51 (m, 1H), 4.31-4.36 (m, 3H), 6.02 (dd, 1H, J=3.2, 6.4), 8.04 (s, 1H), 9.60 (s, 1H); ¹³C NMR (CDCl₃) δ 21.3, 25.1, 33.4, 64.3, 79.8, 87.2, 96.5, 139.6, 149.9, 159.4, 170.9; M+H⁺, C₁₁H₁₄O₅N₂I 380.9942.

(5-(2,4-dioxo-5-(prop-1-ynyl)-3,4-dihydropyrimidin-1(2H)-yl)-tetrahydrofuran-2-yl)methyl acetate—This 5-alkylnyl compound was prepared from 5-iodo compound obtained from last step in 60% yield by the same procedure described above. MP. 162-163° C.; IR (cm⁻¹) 3178 (br), 3088 (br), 1687, 1278; ¹H NMR (400 MHz, CDCl₃) δ 1.77-1.90 (m, 1H), 2.00 (s, 3H), 2.00-2.16 (m, 2H), 2.19 (s, 3H), 2.40-2.50 (m, 1H), 4.29-4.40 (m, 3H), 6.03 (dd, 1H, J=2.8, 6.4), 7.92 (s, 1H), 8.91 (s, 1H); ¹³C NMR (100 MHz, CDCl₃) δ 4.80, 20.93, 25.11, 33.39, 64.28, 70.99, 79.70, 86.92, 90.65, 100.24, 141.90, 149.42, 162.15, 170.88; M+H⁺C₁₄H₁₇O₅N₂ 293.1132.

3-(5-(hydroxymethyl)-tetrahydrofuran-2-yl)-6-methylfuro[2,3-d]pyrimidin-2(3H)-one (14)—To a stirred solution of 5-alkylnyl compound (0.80 g, 2.74 mmol) in methanol and triethylamine 100 ml (7:3) was added copper (I) iodide (0.1 g, 0.55 mmol). The mixture was reflux for 4 h. The solvent was removed in vacuo, and the crude product was purified by flash chromatography (initial eluent: ethyl acetate, followed by ethyl acetate/methanol (9:1). The combined fractions were combined, and the solvent was removed in vacuo to give the crude product, which was recrystallized from methanol to give pure product as white solid (0.31 g, 45%). MP. 130-131° C.; IR (cm⁻¹) 3450 (br), 1666, 1629, 1572, 1180; ¹H NMR (400 MHz, CDCl₃) δ 1.87-1.92 (m, 2H), 2.20 (m, 1H), 2.34 (s, 1H), 2.56-2.63 (m, 1H), 3.83 (dd, 1H, J=4.2, 12), 4.11 (d, 1H, J=12), 4.274 (br, 1H), 6.10 (d, 1H, J=1.2), 6.18 (d, 1H, J=6.0), 8.60 (s, 1H); ¹³C NMR (100 MHz, CDCl₃) δ 14.2, 23.9, 34.1, 62.4, 83.7, 89.2, 100.4, 107.7, 137.1, 155.2, 155.4, 171.8; M+H⁺, C₁₂H₁₅O₄ 251.1024.

3-(5-(hydroxymethyl)-tetrahydrofuran-2-yl)-6-methyl-3H-pyrrolo[2,3-d]pyrimidin-2(7H)-one (25)—To a 40 ml of 7N ammonium in methanol was added compound 14 (80 mg, 0.32 mmol) in a pressure bottle. The mixture was heated to 60° C. for 12 h. The solvent was removed in vacuo, and the crude product was purified by flash chromatography (initial eluent: ethyl acetate, followed by ethyl acetate/methanol (9:1). The combined fractions were combined, and the solvent was removed in vacuo to give the crude product, which was recrystallized from methanol to give pure product (35 mg, 44%). MP. 54-55° C.; IR (cm⁻¹) 3350 (br), 1670, 1560, 1090; ¹H NMR (400 MHz, CDCl₃+a drop of CD₃OD) δ 1.91-1.93 (m, 2H), 2.18 (m, 1H), 2.41 (s, 3H), 2.54 (m, 1H) 3.82 (dd, 1H, J=4.0, 12), 4.08 (dd, 1H, J=2.8, 12.4), 4.26 (m, 1H), 4.80 (d, 1H, 1.6), 6.22 (dd, 1H, J=2.4, 6.8), 8.44 (s, 1H); ¹³C NMR (100 MHz, CDCl₃) δ13.5, 24.2, 33.9, 62.5, 83.2, 88.8, 97.9, 110.6, 134.9, 138.6, 155.5, 158.6; M+H⁺, C₁₂H₁₆O₃N₃ 250.1183.

### Example 2

### Validation of Fluorescent Nucleoside Analogs as Enzyme Substrates

This example presents results of in vitro and in vivo kinase assay testing of some of the exemplary fNAs of the present disclosure.

In Vitro Phosphorylation of fNAs by Kinases

Utilization of the fNAs of the present disclosure by dNKs was also investigated. The phosphorylation of 2′-deoxynucleosides by dNKs represents the initial step in the cellular nucleoside salvage pathway (FIG. 1). As discussed above, the same pathway is also responsible for the activation of nucleoside analog prodrugs and often represents the rate-determining step in the formation of biologically active triphosphate anabolites.

### Deoxynucleoside Kinase Overexpression and Purification

To investigate the kinetic properties of the above-described fNAs in vitro, a pET-based protein overexpression system was established for the production and purification of milligram quantities of individual dNKs. The genes for human 2′-deoxycytidine kinase (dCK) and fruitfly dNK (DmdNK), as well as the thymidine kinase from the bacterium Thermotoga maritime (TmTK) were subcloned into pET-14b (Novagen), an IPTG-inducible protein overexpression system that also introduces a poly-His tag at the target protein's amino terminus. Following protein overexpression, the tag enables one-step purification of the corresponding proteins on Ni-affinity resin, yielding enzyme of >95% purity as determined by SDS-PAGE. This protein overexpression system was successfully applied for the listed wild type kinases above, as well as for isolating engineered enzymes. Separate kinetic experiments confirmed that the poly-His tag does not interfere with kinase activity (data not shown).

### Spectrophotometric Assay for fNA Kinetic Analysis

A quantitative measurement of individual enzymes' substrate specificity is possible via the determination of the kinetic parameters K_{M} and k_{cat}. Consistent with the literature, each kinase will be described by its relative activity for a fNA in comparison to its corresponding NA and thymidine: k_{cat}(fNA)/k_{cat}(NA), as well as by its relative catalytic efficiency: k_{cat}/K_{M}(fNA)/k_{cat}/K_{M}(NA). While these initial kinetic experiments have concentrated on the synthesized fNAs, this characterization can be extended to all fluorophors of the present disclosure.

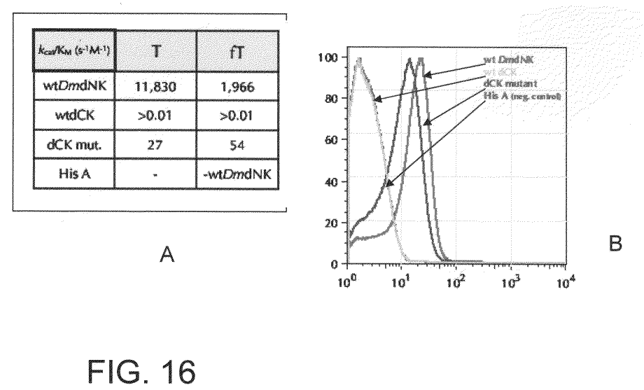

Experimentally, the kinase-catalyzed phosphotransfer reaction was monitored at 37° C. via a spectrophotometric, couple-enzyme assay (as described in Munch-Petersen, B., Knecht, W., Lenz, C., Sondergaard, L. and Piskur, J. (2000) functional expression of a multisubstrate deoxyribonucleoside kinase from Drosophila melanogaster and its C-terminal deletion mutants. J Bio chem, 275, 6673-6679; Schelling, P., Folkers, G. and Schapozza, L. (2001) A spectrophotometric assay for quantitative determination of kcat of herpes simplex virus type 1 thymidine kinase substrates. Anal Biochem, 295, 82-87, which are hereby incorporated by reference herein). The assay is based on a classical enzyme cascade, linking ATP turnover by the deoxynucleoside kinase to NADH reduction via pyruvate kinase and lactate dehydrogenase. The consumption of NADH can be monitored spectrophotometrically at 340 nm. The concentration of all, reactants was adjusted to obtain 20-30% NADH conversion over the 4-minute assay period, guaranteeing linearity throughout the experiment. Three dNKs were tested for their catalytic activity with natural nucleoside thymidine and fluorescent nucleoside fNA 1 (FIG. 10A).

The results presented in FIG. 10A suggest that two of the three type-I dNKs (hdCK, e-hdCK, and DmdNK), which distinguish themselves by their broad substrate specificity, can utilize fNAs at levels similar to the natural nucleosides. DmdNK shows around 10% wild type activity with the corresponding fNAs, while the engineered dCK6mut, a double-mutant (R104Q, D133N) isolated from a random mutagenesis library by selection for thymidine kinase activity, actually displays 2-fold higher activity with the fluorescent substrate than with the natural pyrimidine. The failure of dCK to phosphorylate either substrate can be explained by the enzyme's known intolerance to modifications in the 5-position. The crystal structure of dCK suggests a steric clash between 5-position substituents and the protein's R104 side chain (Sabini, E., Ort, S., Monnerjahn, C. Konrad, M. and Lavine, A. (2003) Structure of human dCK suggests strategies to improve anticancer and antiviral therapy. Nat Struct Biol, 10, 513-519), consistent with the native enzyme's ability to discriminate against thymine substrates and dCKmut's capacity to phosphorylate thymidine.

Additional tests were performed with thymidine kinase from Thermotoga maritima (TmTK), a recently isolated and characterized member of the type-II kinase family. TmTK is structurally and functionally closely related to the human thymidine kinase 1 (hTK1). Members of the type-II family distinguish themselves by their high catalytic turnover of thymidine and 2′-deoxyuridine but complete inactivity for deoxycytidine and the purine deoxynucleosides. Kinetic experiments with compound 1 showed no detectable phosphorylation by type-II kinases, leading to speculation that the modified nucleobase interferes with substrate binding in the enzyme active site. The findings are also consistent with the earlier mentioned inactivity of fluorescent araT against VZV. NA activation in VZV is facilitated by the virus' type-II thymidine kinase, which was predicted to show the same prejudice against fNAs as TmTK. While limiting the use of our current furano pyrimidine-based fNAs, literature reports suggest that type-II kinases do tolerate nucleoside analogs with extensive modifications in the 3-position, offering additional synthetic opportunities for type-II kinase-specific fluorescent substrates.

In Vivo Experiments with Fluorescent Nucleoside Analogs

The visualization of small molecules and their metabolites inside a living cell offers powerful new opportunities for studying biochemical reactions in situ, such as the 5′-phosphorylation of the nucleosides by cellular deoxynucleoside kinases.

As discussed above, uncharged 2′-deoxynucleosides can effectively enter and exit the cellular environment via broad-specificity nucleoside transporter proteins, while the negative charge of one or more phosphate groups in 2′-deoxynucleotides prevent the latter from leaving the cell. Fluorescent NAs that are phosphorylated by deoxynucleoside and deoxynucleotide kinases will become “trapped” inside the cellular compartment, resulting in the increased autofluorescence of these cells. Fluorescence microscopy can be used to qualitatively evaluate the intracellular accumulation and distribution/localization of such a fluorescent probe in single cells. More quantitative, flow cytometry can be employed to assess a cell population and, in combination with fluorescence-activated cell sorting (FACS), isolate subgroups of cells with interesting properties.

Cellular Toxicity of fNAs

A major concern for all in vivo studies with NAs is their toxicity to the host organism. Thus, the cytotoxicity of the compound 1 was evaluated in E. coli cultures at up to 44 μM over 8 hours, twice the working concentration and four times the exposure time that was used in the actual experiments. No changes in bacterial viability or phenotype were detected. Separately, Zhang et al. tested the IC₅₀ of 1 on mammalian cell cultures and found >90% cell survival at 1 mM (72-hour exposure) (Zhang, Jr., et al. (2006), Studies of Nucleoside Transporters Using Novel Autofluorescent Nucleoside Probes. Biochemistry, 45, 1087-1089, which is incorporated herein by reference). While it is believed that the analogues are non-toxic due to substrate discrimination by cellular enzymes (in particular DNA polymerases), further in vitro experiments may be conducted to determine their substrate properties at all stages of phosphorylative activation from deoxynucleoside to dNTP, as well as their incorporation by DNA polymerases.

Monitoring fNAs in Cells by Fluorescence Microscopy

To validate the accumulation of the fluorescent nucleotides inside bacterial cells, a series of fNA-uptake experiments was conducted in two E. coli strains. The E. coli TOP10 (Invitrogen) carries a native type-II thymidine kinase while E. coli KY895 is a thymidine kinase-deficient laboratory strain. When incubated with fNA 1, both strains show no fluorescence signal beyond the cellular autofluorescence, consistent with the above preliminary in vitro experiments which show that type-II kinases cannot phosphorylate compound 1.

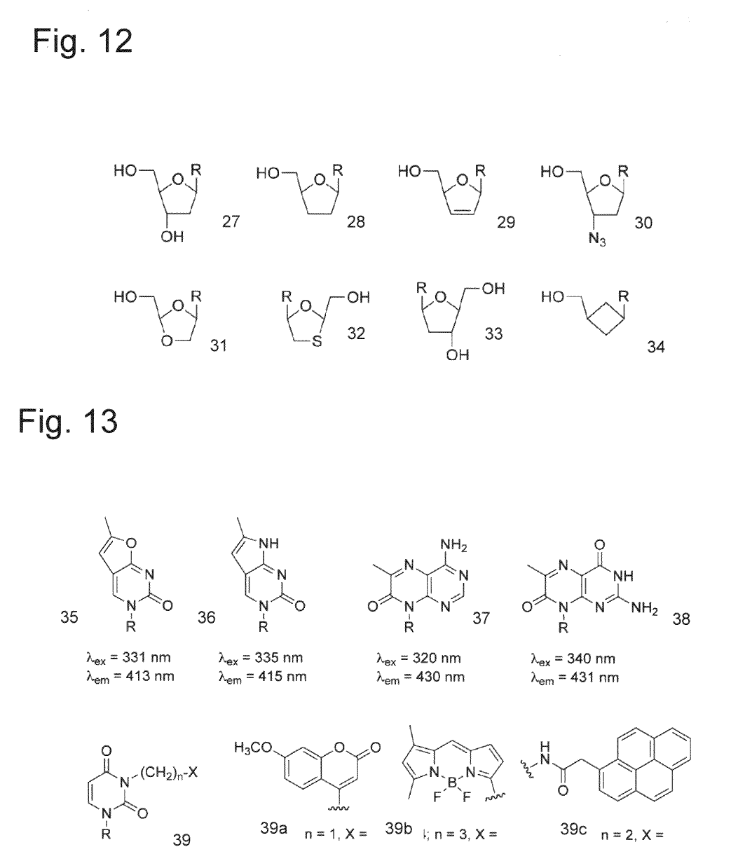

In contrast, when the same bacteria expressed a plasmid-encoded exogenous type-I kinase such as DmdNK, fluorescence microscopy revealed the accumulation of the fluorescent probe inside both strains (FIG. 8). FIG. 8 illustrates the fluorescence microscopy of E. coli KY895 with pDIM-tDmdNK (left) and TOP10 with pBAD-tDmdNK (right) in the presence of 1 (100× magnification; excitation: Hg-lamp at 325 nm). Noticeable in the two images of FIG. 8 is the highly variable fluorescence intensity in the E. coli KY895/pDIM sample, caused by disproportionate expression levels of the lac-promoter system. On the contrary, the arabinose-controlled pBAD system in E. coli TOP10 facilitates steadier kinase expression levels, resulting in a more uniform fluorescence signal. In summary, these experiments provide evidence for the cell's ability to phosphorylate fNAs and accumulate the charged reaction product in quantities significantly above the cellular autofluorescence background.

FACS-based screening of deoxynucleoside kinases for NA Activation

Fluorescent NAs represent a powerful new tool for a variety of studies on nucleoside transport across the membrane and their metabolism in the host cell. Besides possible studies of their in situ biochemistry, the present disclosure provides methods of using fNAs as a new and efficient screening method for engineered deoxynucleoside kinases with catalytic activity for specific NA prodrugs. As mentioned above, existing methods for the identification of NA-activating kinases in combinatorial libraries are limited to the selection of engineered enzymes via genetic complementation in E. coli KY895 or screening for cytotoxicity upon replica-plating on NA-containing media.

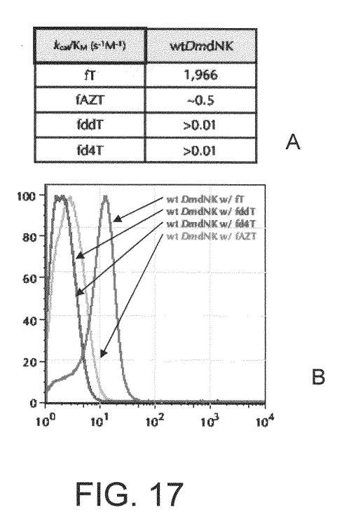

The combination of fNAs with flow cytometry now offers a novel high-throughput screening technique for the evaluation of large combinatorial libraries of engineered deoxynucleoside kinases (FIG. 11). Mutations in kinases that lead to more efficient phosphorylation of individual fNAs result in stronger fluorescence of the host organism. Flow cytometry enables a rapid global analysis of the fluorescence in a bacterial population and, with the help of an attached cell sorter, the separation and collection of highly fluorescent cells from the culture.

In a typical flow cytometry experiment, bacterial cultures are grown to mid-log phase and protein expression is induced for 4 h, followed by the addition of the fN or fNA to the culture broth. Cells are incubated for another 2 h prior to centrifugation and three wash-cycles with PBS buffer to reduce the fluorescence background. Cultures are then analyzed on a Becton Dickinson FACSVantage SE, equipped with a UV laser.

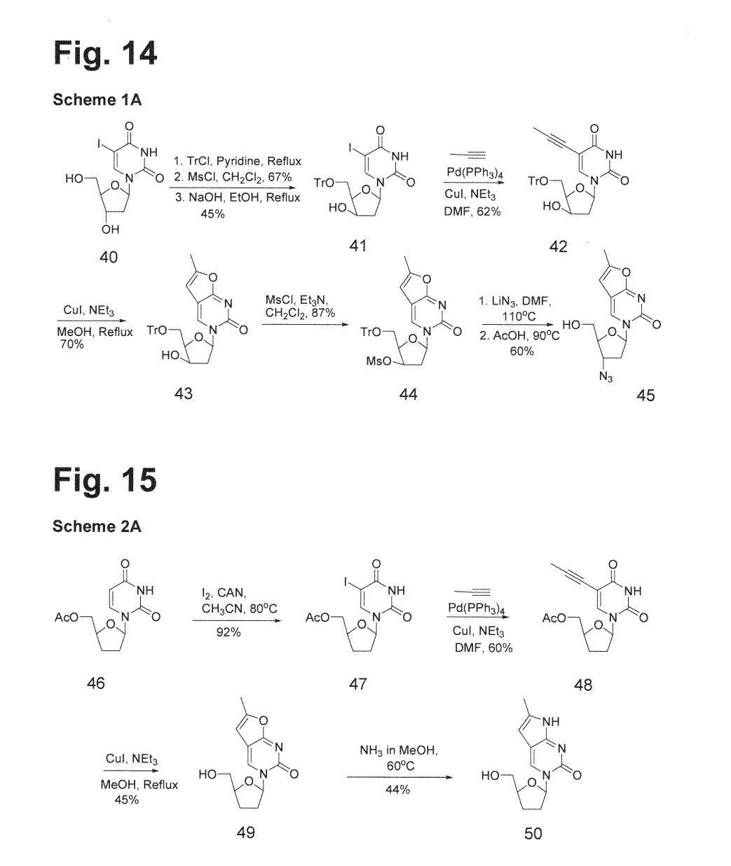

Several experiments were conducted to validate the design for a FACS-based high-throughput screening system for engineered dNKs. A first set of experiments addressed three main questions: a) what is an appropriate host/expression system for kinases in conjunction with fNAs (FIG. 9), b) are the fluorescent properties of the above-described fNAs sufficient for FACS, and c) what is the dynamic range of this screening system? To investigate these questions, testing was performed with three DNA plasmid vectors and their corresponding E. coli host strains: the pDIM-vector (as described in Lutz, S. et al (2001) Nucleic Acids Research vol. 29 iss. 4 page e16, which is incorporated herein by reference), a low-copy number plasmid that places the kinase under control of a lac promoter in E. coli KY895, the arabinose-controlled, high-copy pBAD-vector in E. coli TOP10 (Invitrogen), and the lac promoter-based, high-copy pET-vector in E. coli BL21(DE3) (Novagen), with compound 1 as the fluorescent substrate. The results in FIG. 9 are consistent with the microscopy data described above, showing that only a very small fraction of the pDIM-carrying culture overexpresses DmdNK, resulting in a small subpopulation with fluorescence above background. In contrast, both the pET and pBAD system yield populations with a more significant portion of “positives”, cells with 10 to 20-fold higher fluorescence than background.

The pBAD system was further tested with three type-I kinases, DmdNK, dCK and dCKmut (FIG. 10B) to explore the fluorescence signature of kinases with various catalytic activities. The fruit fly enzyme shows approximately 500-times higher catalytic activity than dCKmu7t, probing the dynamic range of the selection system over two to three orders of magnitude. In contrast, hdCK shows no significant thymidine kinase activity and serves as negative control.

We also evaluated the substrate promiscuity of wild type DmdNK, the kinase with very high activity and the broadest substrate specificity towards our fluorescence nucleoside analogs. FIG. 11A lists the kinetic properties of the enzyme for the various substrates and FIG. 11B shows the corresponding FACS histograms. The two dataset are in good agreement as DmdNK shows strong activity for 1, only background phosphorylation of 16, and no detectable activity for 25 and 26. These data again demonstrate the good correlation of in vitro and in vivo data.

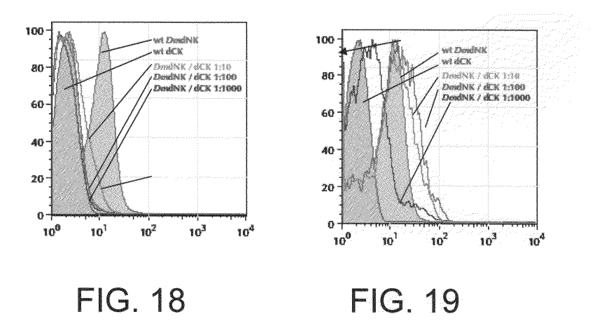

Finally, we determined the enrichment factor in artificial libraries consisting of known mixing ratios of DmdNK and dCK. The former exemplifies a positive (functional) library member while the latter represents the negative (inactive) library members. Mixtures at ratios of 1:10 (one mole equivalent of DmdNK in ten mole equivalent of dCK) to 1:1000 (one mole equivalent of DmdNK in a thousand mole equivalent of dCK) were analyzed by FACS (FIG. 12A), followed by gated sorting for members with high fluorescence. After sorting, the collected high-fluorescence fraction was re-analyzed (FIG. 12B) and showed complete enrichment of DmdNK in the 1:10 and 1:100 mixture while the 1:1000 experiment retained a substantial fraction of dCK-carrying cells. In summary, these experiments suggest that the FACS sorting allows for a 100 to 1000-fold library enrichment.

### Conclusions

The application of fNAs as molecular probes provides a powerful new tool for studying the uptake and metabolism of antiviral prodrugs. The examples above demonstrate the successful synthesis of novel fluorescent pyrimidine prodrugs that showed substrate properties similar to the natural nucleosides in assays with type-I dNKs. Separately, fNAs were tested in bacterial cultures, taking advantage of intracellular fluorophor accumulation as a result of phosphorylation. The same feature can be utilized to assay large combinatorial libraries of dNKs, identifying and isolating enzymes with activity for NA prodrugs by FACS. The latter high-throughput screen represents a significant improvement over previous selections and screening techniques, allowing for the first time the direct positive selection for fNA phosphorylation activity. These examples conclusively demonstrate the successful identification of mutant kinases with novel activity.

### Example 3

### Application of Fluorescent Nucleoside Analogs for the Identification of Novel Deoxynucleoside Kinases Via Directed Evolution

The procedures for the present Example include monitoring fNAs in cells by fluorescence microscopy and FACS-based screening of deoxynucleoside kinases for NA activation, similar to procedures described above in Example 2, with modifications for different dNKs or fNAs of interest. Additional details and results are discussed below.

Materials: 6-methyl-3-(β-D-2-deoxyribofuranosyl)furano-[2,3-d]pyrimidin-2-one (1) was purchased from Berry & Associates (Dexter, Mich.). All primers for cloning were from Integrated DNA Technologies (Coralville, Iowa). Pfu Turbo DNA polymerase (Strategene, La Jolla, Calif.) was used for all, cloning. Restriction enzymes were from New England Biolabs (Beverly, Mass.) unless otherwise indicated. Pyruvate kinase and lactate dehydrogenase were from Roche Biochemicals (Indianapolis, Ind.). Ampicillin (Amp) and Chloramphenicol were from Fisher Scientific (Fair Lawn, N.J.). All other reagents were from Sigma-Aldrich (St. Louis, Mo.) unless otherwise indicated.

Construction of libraries: Random mutagenesis libraries were created using error-prone PCR introducing mutations into a truncated form of the dmdnk gene (tdmdnk) gene, which is the wild-type gene with 10 residues after start codon at the N-terminus and 15 residues before the stop codon at the C-terminus removed. The tDmdNK was confirmed to have similar activity to the wild-type. GeneMorph II random mutagenesis kit (Strategene, La Jolla, Calif.) was used with an average of two mutations for each generation. Plasmid-specific primers flanking the gene were used for the PCR (forward primer: ATG CCA TAG CAT TTT TAT CC-3′ (SEQ ID NO: 1); reverse primer: 5′-GAT TTA ATC TGT ATC AGG-3′ (SEQ ID NO: 2)). The gene libraries were cloned into pBAD-HisA (Invitrogen, Carlsbad, Calif.) between Nco I and Hind III sites. The cloned products were transformed into E. coli TOP10 cells (Invitrogen, Carlsbad, Calif.), plated on LB (Amp, 50 μM) plates, and colonies were collected from plates and aliquots stored at −80° C. Mutation frequencies were confirmed by DNA sequencing. Three generations of random mutagenesis libraries were constructed. Each generation was used as templates for the subsequent generation.

A DNA shuffling library was made using the Nucleotide exchange and excision technology (NExT) as described in Kristian M. Müller, S. C. S., Susanne Knall, Gregor Zipf, Hubert S. Bernauer, Katja M. Arndt, Nucleotide exchange and excision technology (NExT) DNA shuffling: a robust method for DNA fragmentation and directed evolution, Nucleic Acids Res., 2005. 33(13): p. e117, which is incorporated herein by reference. The gene pool sorted from the third generation random mutagenesis library (ep_Lib3^{rd}) and tdmdnk gene were PCR amplified separately using gene-specific primers containing Nco I and Hind III cleavage sites (forward primer: 5′-CCG CCA TGG GGA AGT ACG CCG AGG GCA CC-3′ (SEQ ID NO: 3); reverse primer: 5′-CCC AAG CTT CAG GGC TGT TGG TTA CTT GA-3′ (SEQ ID NO: 4)). A dNTP mixture (0.2 mM dA/G/CTP each, 0.066 mM dUTP and 0.134 mM TTP) was used in amplification with Taq DNA polymerase. The PCR products were then agarose gel-purified and incubated with uracil-DNA-glycosylase (UDG) at 37° C. for one hour to cleave out the incorporated uracil. 10% Piperidine was added and the mixture was incubated at 90° C. for 30 min to cleave the DNA. The fragments were then run on 2% low-melt agarose gel (Cambrex Bio Science Rockland, Rockland, Me.) and purified using QIAEX II gel extraction kit (QIAGEN, Valencia, Calif.). The purified DNA were mixed together at 20:1 (mutant pool:wild-type) ratio and reassembled through PCR using the following conditions: 95° C. for 2 min; 45 cycles of 94° C. for 15 sec, 1° C./s ramp to 50° C., 50° C. for 1 min, 72° C. for 1 min+4 s/cycle; one cycle of 72° C. for 7 min. The PCR product was agarose gel-purified and cloned into pBAD-HisA between Nco I and Hind III sites, followed by transformation into E. coli TOP10 cells, plated on LB (Amp, 50 μM) plates, and colonies were collected from plates and stored at −80° C.

A site saturation mutagenesis library at Glu172 and Tyr179 was introduced by overlap extension PCR using a degenerate primer with NNS (N: 25% A/T/C/G; S: 50% G/C) codon for the two residues. The overlapping primers are: forward primer 5′-CGG GCT CGT TCT GAG NNS AGC TGC GTG CCG CTT AAG NNS CTT CAG GAG C-3′ (SEQ ID NO: 5), reverse primer: 5′-CTC AGA ACG AGC CCG CTG-3′ (SEQ ID NO: 6). The primers were used first with a gene-specific primer at each terminus to produce two fragments. These fragments were combined using overlap PCR to produce the full-length gene with saturated mutations at both 172 and 179. The gene was inserted in pBAD-HisA and transformed into E. coli TOP10 cells, plated on LB (Amp, 50 μM) plates, and colonies were collected from plates and stored at −80° C. DNA sequencing of library members confirmed an even distribution of residues at the desired positions.

FACS sorting: To prepare cells for sorting, 2 ml LB (Amp, 50 μM) was inoculated with a stab of cells from freezer stock and grown for 13 hr at 37° C., and then 40 μl of that was further used to inoculate a 4 ml LB (50 μM amp). Arabinose was added to the media (0.2%, final concentration) when OD₆₀₀ reached ˜0.5. Culture grew for 4 more hours before fluorescent NA was added and incubated for another 30 min to 2 hr (details described in table 1.). Cells were harvested by centrifugation (800 g, 10 min, 4° C.) followed by washing with PBS buffer (pH 7.4) three times. Lastly, the cells were resuspended in PBS (pH 7.4) to 1×10⁸ cells/ml for sorting.

TABLE 1
Conditions of preparing cells for sorting

| | | Incubation | |
|---|---|---|---|
| Libraries | Amount of fddT and Thymidine | time | |
| ep_Lib1st | fddT (40 μM) | 2 | h |
| ep_Lib2nd | fddT (80 μM) | 2 | h |
| ep_Lib3rd 1^{st} sort | fddT (80 μM) | 1 | h |
| 2^{nd} sort | fddT (80 μM) + Thymidine (800 μM) | 30 | min |
| SH_Lib | fddT (5 μM) + Thymidine (50 μM) | 30 | min |
| SM_Lib | fddT (5 μM) + Thymidine (50 μM) | 15 | min |

Sorting was done with the FACSVantage (Becton Dickinson, Franklin Lakes, N.J.). The UV laser was used for excitation, and a band pass filter (424±20 nm) was used for emission detection. Sorting was performed in standard mode on ˜10⁷ events at a speed of less than 2,000 events/s. Gate for sorting was set depending on the difference between the sample library and negative control. For each library, sorting was done in triplicate: Cells were collected into SOC and incubated at 37° C. for 2-hr before being spread on a LB-Agar plate (Amp, 50 μM). 10 colonies were picked for sequencing and further characterization and the rest were harvested and serve as template for subsequent generation.

Over-expression and purification: Genes of interest were cloned into pMAL-C2x (New England Biolabs, Beverly, Mass.), then transformed into E. coli BL21(DE3)pLySs. Expressions were performed at 37° C. A single colony was picked for 2 ml overnight culture of LB media (100 μM Amp, 34 μM chloramphenicol and 0.2% glucose). Then 100 ml LB media with same recipe was inoculated with 1 ml of the starting culture. Isopropyl-β-D-1-thiogalactopyranoside (IPTG) was added (0.3 μM, final concentration) when OD₈₀₀ reached ˜0.5. The expression culture grew for another 4 hr before cells were harvested by centrifugation (4000 g, 20 min, 4° C.). Cell pellets were resuspended in 5 ml Column Buffer (20 mM Tris-HCl pH 7.4, 300 mM NaCl, 1 mM EDTA) and stored at −20° C. overnight.

Cell pellets were thawed in an ice-water bath and sonicated 8 times (pulse times: 10 sec on/10 sec off). Supernatant was collected after centrifugation at 9,000 g for 30 min at 4° C. and mixed with pre-washed amylase resin (New England Biolabs, Beverly, Mass.) and incubated for 1 hr. The resin was then loaded to Poly-Prep Chromatography columns (0.8×4 cm, Bio-Rad, Hercules Calif.), and proteins were eluted with 3 fractions of 600 μl Elution Buffer (Column Buffer+10 mM maltose). Eluants were analyzed via SDS-PAGE gel. The fractions containing proteins were pooled and concentrated to 3-4 mg/ml in Amicon-Ultra centrifugal filter units (Amicon Bioseparations, Billercia, Mass.). Protein concentrations were quantified by measuring A₂₈₀ (MBP-tDmdNK, ξ=106,230 M⁻¹cm⁻¹, calculated according to Pace et al., How to measure and predict the molar absorption coefficient of a protein, Protein Sci, 1995. 4(11): p. 2411-2423, which is incorporated herein by reference). Protein aliquots were stored at −80° C.

Steady-state kinetic assays: Activity for nucleosides and NAs. Assays were carried on at 37° C., in a 500 μl reaction mixture containing 50 mM Tris-HCl (pH 8.0), 300 mM NaCl, 2.5 mM MgCl₂, 0.18 mM NADH, 0.21 mM phosphoenolpyruvate, 1 mM ATP, 1 mM 1,4-dithio-DL-threitol, 30 U/ml pyruvate kinase, 33 U/ml lactate dehydrogenase, and substrate with concentrations ranging from 1 μM to 7 mM. Measurements were made in triplicate and corrected for background. Data were fit to the Michaelis-Menten equation using Origin (OriginLab, Northhampton, Mass.) to get apparent. Kₘ and Vₘₐₓ, and then k_{cat} is calculated from Vₘₐₓ.

### Example 4

### Fluorescent Substrates for Type-II Kinases

The problem with type-II kinase substrate specificity may be addressed with compounds and methods of the present disclosure. As discussed above, the furano-pyrimidines are not recognized by this kinase subfamily, presumably due to steric limitations in the substrate-binding site. At the same time, reports of efficient catalytic turnover of nucleoside analogs with modifications in the 3-position suggest that alternative fluorophor may be utilized by these enzymes. These synthetic efforts would be limited to thymine nucleosides as type-II kinases do not process the other three natural 2′-deoxyribonucleosides. Besides broadening and diversifying our fNA library, a second category of thymidine fluorophors might also enable us to selectively monitor the phosphorylation activity of individual kinase subfamilies in vivo.

Inspiration for the synthesis of such second-generation fNAs can be drawn from studies of 2′-deoxy uridines with substitutions in the 3- and 5-position of the nucleobase, which indicated that 3-substituted carboranyl nucleosides are good substrates for hTK1 while 5-substitutions are not well tolerated. An impressive example demonstrating the flexibility and tolerance for bulky substituents in 3-modified thymidines are carborane cages. Enzymatic tests showed that these boronated nucleoside analogues were hTK1 substrates at 25-50% the activity for thymidine. Related studies also addressed concerns of steric hindrance and solubility problems in connection with the bulky, hydrophobic side chain, as well as optimal linker length between nucleoside and fluorophor. (Johnsamuel, J., et al. (2004) Synthesis of Ethyleneoxide Modified 3-carboranyl Thymidine Analogues and Evaluation of Their Biochemical, Physicochemical, and Structural Properties. Bioorganic & Medicinal Chemistry, 12, 4769-4781; Byun, Y., et al. (2005) Synthesis and Biological Evaluation of Neutral and Zwitterionic 3-carboranyl Thymidine Analogues for Boron Neutron Capture therapy. J Med Chem, 48, 1188-1198, which are incorporated herein by reference).

In addition to 3-carboranyl modifications of thymidine and uridine, a wide range of 3-substituents including fluorophors 7 and 9 have been reported, but their nucleoside kinase substrate properties are unknown. Additional fNAs, such as 3-(methoxy-coumarin)-thymidine (FIG. 3, compound 7), 3-(BODIPY)-thymidine (FIG. 3, compound 8), and 3-(pyrene)-thymidine (FIG. 3, compound 9) can be synthesized and their cellular uptake and metabolism tested in vitro and in vivo as outlined above, with special emphasis put on the evaluation of these compounds with type-II kinases.

The synthesis for 7-9 will largely follow standard protocols. While 7 can be prepared by reaction of thymidine with 4-bromomethyl-methoxy coumarin (Sigma-Aldrich) (as described in Use of 4-Bromomethyl-7-Methoxycoumarin for Derivatization of Pyrimidine Compounds in Serum Analyzed by High-Performance Liquid-Chromatography with Fluorometric Detection. Yoshida, S., et al. (1986) Journal of Chromatography, 383, 61-68, which is incorporated herein by reference), the borano-fluorophor 8 is accessible via reduction of the corresponding carboxylate (Molecular Probes; Eugene, Oreg.) and coupling of its tosylate intermediate to thymidine. Finally, the pyrene derivative will be linked to thymidine via an ethylenediamine linker (as described in Multiple-pyrene Residues Arrayed Along DNA Backbone Exhibit Significant Excimer Fluorescence. Kosuge, Mr., et al. (2004) Tetrahedron Letters, 45, 3945-3947, which is incorporated herein by reference). Successful phosphorylation of the 2′-deoxyribose analogs by type-II kinases will lead to the preparation of the corresponding modified sugar derivatives discussed above.

We claim:

1. A fluorescent nucleoside analog (fNA) comprising:
a fluorescent nucleobase selected from one of the following: 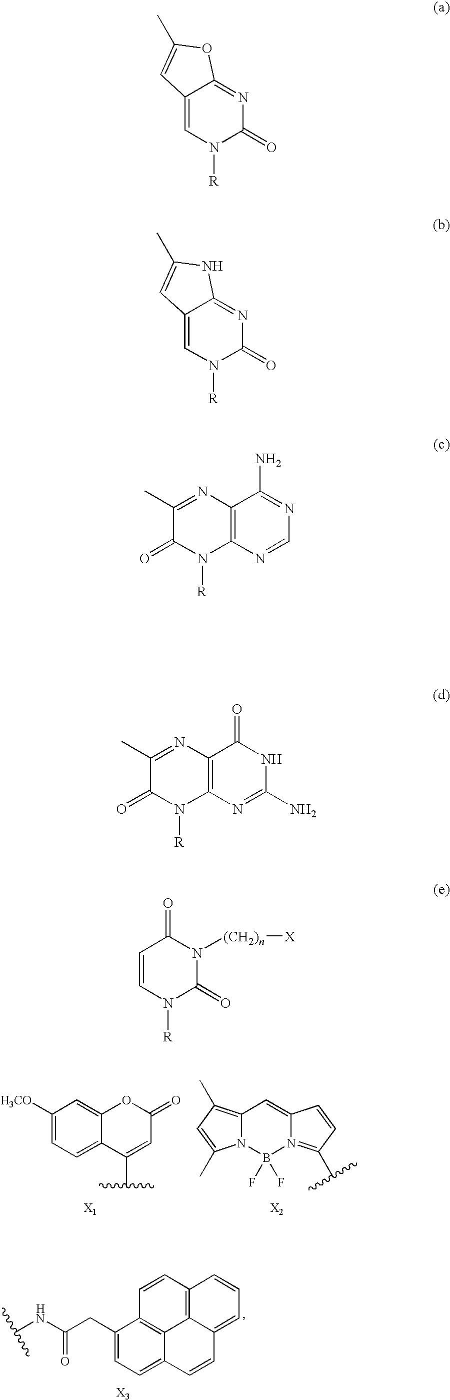
wherein, for fluorescent nucleobase (e), X is selected from X₁, X₂ and X₃; and
a modified sugar moiety selected from one of the following: 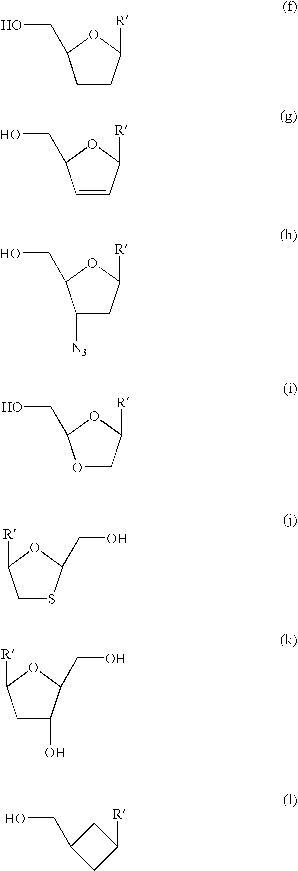
wherein for fluorescent nucleobases (a)-(e), R represents one of the modified sugar moieties (f)-(l) and for modified sugar moieties (f)-(l), R' represents one of the fluorescent nucleobases (a)-(e), and
wherein the fNA is capable of producing a detectable fluorescent signal.

2. The fNA of claim 1, wherein the fNA is a fluorescent variant of a nucleoside analog prodrug used to treat a viral disorder.

3. The fNA of claim 1, wherein the fluorescent nucleobase is a fluorescent furano-pyrimidine base.

4. The fNA of claim 1, wherein the fluorescent nucleobase is a fluorescent pyrrolo-pyrimidine base.

5. The fNA of claim 1, wherein the fluorescent nucleobase is a fluorescent pterine base.

6. A method of detecting in vivo phosphorylation of a fluorescent nucleoside analog (fNA) comprising:
contacting a cell with a composition comprising a plurality of at least one type of fNA, and
detecting a change in fluorescence intensity inside the cell, wherein an increase in fluorescence intensity inside the cell indicates phosphorylation of the fNA.

7. The method of claim 6, wherein detecting an increase in fluorescence intensity in the cell indicates accumulation of fNAs inside the cell, indicating the phosphorylation of the fNA.

8. The method of claim 6, further comprising detecting the distribution of the fNAs inside the cell.

9. The method of claim 6, wherein fluorescence microscopy is used to detect the change in fluorescence intensity.

10. The method of claim 6, further comprising contacting a population of cells with the composition of fNAs, and detecting and isolating cells with increased fluorescence, wherein increased fluorescence indicates phosphorylation of the fNA.

11. The method of claim 10, wherein fluorescence-activated cell sorting (FACS) is used to detect and isolate the cells with increased fluorescence.

12. A method of screening for kinases capable of phosphorylating a fluorescent nucleoside analog (fNA) comprising:
providing at least one cell that over-express a kinase of interest;
providing a composition comprising at least one fNA;
contacting the cell with the composition fNA; and
detecting a change in fluorescence intensity inside the cell, wherein an increase in fluorescence intensity indicates phosphorylation of the fNAs by the kinase

13. The method of claim 12, wherein the kinase is a deoxynucleoside kinase.

14. The method of claim 12, wherein the kinase is a modified deoxynucleoside kinase.

15. The method of claim 12 further comprising:
separately contacting at least one cell that over-express the kinase of interest with fluorescent analogs of natural nucleosides, detecting a change in fluorescence intensity inside the cell, and comparing the change in fluorescence intensity detected with the fluorescent analog of the natural nucleoside to the change in fluorescence intensity detected with the fNA.

16. The method of claim 12, further comprising:
providing a population of cells expressing a kinase of interest, wherein the population of cells includes at least two subpopulations of cells, wherein each subpopulation of cells expresses a different kinase of interest;
and detecting the cells with increased fluorescence, indicating the phosphorylation of the fNA by the kinase of interest expressed by that cell.

17. The method of claim 16, wherein fluorescence-activated cell sorting (FACS) is used to detect and isolate cells with increased fluorescence.

18. A method of screening for kinases capable of phosphorylating a fluorescent nucleoside analog (fNA) comprising:
providing a population of cells expressing a variety of kinases;
providing a composition comprising at least one fNA;
contacting the population of cells with the fNA composition;
detecting the cells having an accumulation of fNAs inside the cell, indicating the phosphorylation of the fNA by a kinase expressed by that cell.

19. The method of claim 18, wherein fluorescence-activated cell sorting (FACS) is used to detect and isolate cells with increased fluorescence.

20. The method of claim 18, wherein fluorescence-activated cell sorting (FACS) is used to screen large samples of cells for cells with pre-determined levels of fluorescence

21. The method of claim 18, further comprising identifying and isolating kinases from the cells having an accumulation of fNAs inside the cell.


SEQUENCE LISTING (6 sequences)

SEQ ID NO: 1 - DNA, 20 bp, Artificial Sequence
  Chemically synthesized forward primer
atgccatagcatttttatcc

SEQ ID NO: 2 - DNA, 18 bp, Artificial Sequence
  Chemically synthesized reverse primer
gatttaatctgtatcagg

SEQ ID NO: 3 - DNA, 29 bp, Artificial Sequence
  Chemically synthesized forward primer
ccgccatggggaagtacgccgagggcacc

SEQ ID NO: 4 - DNA, 29 bp, Artificial Sequence
  Chemically synthesized reverse primer
cccaagcttcagggctgttggttacttga

SEQ ID NO: 5 - DNA, 49 bp, Artificial Sequence
  Chemically synthesized forward primer sequence
cgggctcgttctgagnnsagctgcgtgccgcttaagnnscttcaggagc

SEQ ID NO: 6 - DNA, 18 bp, Artificial Sequence
  Chemically synthesized reverse primer
ctcagaacgagcccgctg